(19) United States
(12) Patent Application Publication
(10) Pub. No.: US 2025/0043308 A1
(43) Pub. Date: Feb. 6, 2025

(54) Purification Method of Viral Vectors

(71) Applicant: Oxford Biomedica (UK) Limited, Oxford (GB)

(72) Inventors: Lee Davies, Oxford (GB); Oliver Goodyear, Oxford (GB); Samuel Petrucci, Oxford (GB); Maria Kapanidou, Oxford (GB); George T. Pamenter, Oxford (GB); William Bowen, Oxford (GB)

(21) Appl. No.: 18/718,051

(22) PCT Filed: Dec. 8, 2022
(86) PCT No.: PCT/GB2022/053136

§ 371 Date: Jun. 9, 2024

(30) Foreign Application Priority Data
Dec. 9, 2021 (GB) 2117844.7

(51) Int. Cl. C12N 15/86; B01D 15/18; B01D 15/36; B01D 15/42; B01D 61/14

(52) U.S. Cl. CPC C12N 15/86; B01D 15/1871; B01D 15/362; B01D 15/363; B01D 15/424; B01D 61/14; B01D 2315/10; C12N 2740/15043; C12N 2740/15051; C12N 2740/16043; C12N 2740/16051

(57) ABSTRACT

A method of purifying a viral vector preparation is described. The method comprises: passing a viral vector preparation through a cation exchange column, contacting flow-through from the cation exchange column with an anion exchanger that binds the viral vector, and eluting the bound viral vector from the anion 5 exchanger as a viral vector eluate.

### BACKGROUND

Viral vectors have been increasingly used as a tool for gene and cell therapies. In particular, there has been considerable interest in lentiviral vectors because of their ability to infect both dividing and non-dividing cells.

Various methods of viral vector manufacture are known. For example, suitable methods include the transfection of primary cells or mammalian/insect cell lines with vector DNA components, followed by a limited incubation period and then harvest of crude vector from culture media and/or cells (Merten, O-W., Schweizer, M., Chahal, P., & Kamen, A. A., 2014, Pharmaceutical Bioprocessing, 2:183-203). In other examples, producer cell lines (PrCLs; where all of the necessary vector component expression cassettes are stably integrated into the production cell DNA) are used during transfection-independent approaches, which is advantageous at larger scales.

Methods of purifying viral vector preparations are also known. For example, for laboratory applications, viral vector preparations may be purified using simple centrifugation techniques. However, to be used in clinical protocols, viral vector preparations may need to comply with different standards of, for example, purity and titre. In recent years, various downstream processing steps (DSP's) have been designed to achieve desirable recovery yields of viral vectors, for example, with defined attributes that can affect the safety and efficacy of the final product.

### BRIEF DESCRIPTION OF THE DRAWING

Features of examples of the present disclosure will become apparent by reference to the following detailed description and drawings:

FIG. 1 is a flowchart 10 of an example of a prior art process for purifying a viral vector preparation (Process A);

FIG. 2 is a flowchart 100 of an example of a modified process for purifying a viral vector preparation (Process B);

FIG. 3 is a flowchart 200 of an example of a process for purifying a viral vector preparation in accordance with an example of the present invention (Process C);

FIGS. 4A and 4B show particle size measurements made using dynamic light scattering quantification (DLS) to demonstrate the impact of Salt Activated Nuclease (SAN) (Process B) on reducing particle size and overall aggregation compared to Benzonase® (Process A);

FIG. 5 compares the impact of Processes A and B on log reduction of total residual DNA and functional vector titre recovery following sterile filtration;

FIG. 6 compares the impact of Processes A and B on membrane pressure;

FIG. 7 shows how Processes B and C impact particle size as measured by DLS.

FIG. 8 compares the pressure profiles across the membranes used in the buffer exchange step 20 in Processes A, B and C.

FIG. 9 shows the total DNA reduction achieved using Processes A, B and C; and

FIG. 10 compares the mass spectrometry analysis of vector purified using either Process C or Process B.

### DETAILED DESCRIPTION

According to a first aspect, there is provided a method of purifying a viral vector preparation. The method comprises passing a viral vector preparation through a cation exchanger, contacting flow-through from the cation exchanger with an anion exchanger that binds the viral vector, and eluting the bound viral vector from the anion exchanger as a viral vector eluate.

It has been found that, by passing a viral vector preparation through a cation exchanger and contacting flow-through from the cation exchange column with an anion exchanger, improved separation of e.g. DNA impurities from the host cell can be achieved. Because of negative charge(s) on its surface, DNA impurities can sometimes bind to positively charged sites of anion exchangers. As a result, purification of viral vector preparations by anion exchange can result in co-purification of DNA contaminants together with the viral vector. Separation of the DNA from co-eluted viral vector may be difficult, particularly if the DNA becomes associated or complexed with species to form aggregates or agglutinates. Such aggregates or agglutinates can be retained by downstream filtration membranes together with the viral vector. Furthermore, such aggregates or agglutinates can shield the DNA from, for example, downstream nucleic acid cleavage reactions, making it more difficult for the DNA contaminants to be cleaved and/or separated from the viral vectors during subsequent filtration.

It has now been found that DNA may interact or form complexes with e.g. positively charged species in the viral vector preparation, including, for example, histones. This interaction and/or complex-formation may allow DNA-containing aggregates or complexes to bind to the negative sites on a cation exchanger. This can facilitate separation of at least some DNA from the viral vector preparation by cation exchange, such that the flow-through that is subsequently treated by anion exchange contains reduced levels of DNA.

It has also been found that, despite their positive charge, some positively charged contaminants e.g. histones and other DNA and/or RNA binding proteins can be co-eluted with the viral vector during anion exchange. The reasons for this are not well understood. However, histones, for example, may form complexes, aggregates and/or agglutinates with negatively charged species, such as DNA, and the resulting species may have negative surface charges that facilitate their removal by anion exchange. By passing a viral vector preparation through a cation exchanger, the concentration of positively charged contaminants, such as histones, may be reduced prior to anion exchange. As histones can form particle aggregates, the removal of histones and other positively charged species by cation exchange can improve the efficacy of any downstream filtration steps. Excessive particle aggregation can be detrimental to the throughput of any downstream filtration steps, for example, by prematurely restricting or blocking the pores of filtration membranes.

Any suitable cation exchanger may be employed. For example, the cation exchanger may comprise a strongly acidic cation exchanger. Preferably, the cation exchanger may comprise a sulfonic acid cation exchanger. The cation exchanger may be a cation exchange membrane.

Any suitable anion exchanger may be employed. For example, the anion exchanger may be a strongly basic anion exchanger. Preferably, the anion exchanger comprises a quaternary ammonium anion exchanger. The anion exchanger may be an anion exchange membrane.

The viral vector may be selected from the group consisting of: a retroviral vector, an adenoviral vector, an adeno-associated viral vector, a herpes simplex viral vector, a vaccinia viral vector, a picornaviral vector, and an alphaviral vector. Preferably, the viral vector is a retroviral vector. More preferably, the retroviral vector is a lentiviral vector. In some examples, the lentiviral vector may be derived from a HIV-1, HIV- 2, SIV, FIV, BIV, EIAV, CAEV or visna lentivirus.

The viral vector preparation may be produced using any suitable method. Preferably, the viral vector preparation is produced by culturing cells that produce the viral vector and harvesting a viral vector-containing supernatant from the cell culture. Preferably, the supernatant harvested from the cell culture is clarified. The clarified supernatant may be used as the viral vector preparation that is passed through the cation exchanger. In some examples, nucleic acid cleavage may be performed on the cell culture prior to clarification.

The viral vector preparation may comprise non-viral vector DNA, for example, DNA from the host cell. Non-viral vector DNA is used to refer to any DNA that is not comprised within the viral vector. For example, host cell DNA or any DNA that is not part of a viral vector genome packaged within the viral vector. Throughout the description and unless otherwise specified, the term DNA used in relation to a viral vector preparation is used to refer to non-viral vector DNA. The DNA may be a contaminant. At least some DNA may be bound to the cation exchange column when the viral vector preparation is passed through the cation exchange column.

As mentioned above, DNA may interact or form complexes with positively charged species in the viral vector preparation, including, for example, histones. The viral vector preparation may comprise histones and/or DNA, for example, histones and DNA. At least some DNA and/or histones may be bound to the cation exchanger when the viral vector preparation is passed through the cation exchange column. In some examples, at least some DNA and at least some histones may be bound to the cation exchanger when the viral vector preparation is passed through the cation exchanger. For example, the DNA may form a DNA-histone complex. At least some of these complexes may be bound to the cation exchanger when the viral vector preparation is passed through the cation exchanger.

Preferably, the flow-through from the cation exchanger may be contacted directly with the anion exchanger. Alternatively, the flow-through from the cation exchanger may be treated prior to contact with the anion exchanger. For example, the flow-through from the cation exchanger may be subjected to nucleic acid cleavage prior to contact with the anion exchanger.

Preferably, the viral vector eluate from the anion exchanger is subjected to nucleic acid cleavage. Nucleic acid cleavage may be employed to degrade, for example, DNA (e.g. from the host cell). Any suitable method of nucleic acid cleavage may be employed. For example, nucleases, such as endonuclease may be employed. An example of an endonuclease is Benzonase® Nuclease. Preferably, however, the viral vector eluate is treated using a halotolerant and/or halophilic nuclease (e.g. endonuclease). An example of a suitable halotolerant and/or halophilic nuclease may be Salt Active Nuclease (SAN) and M-SAN HQ supplied by ArticZymes®. Another example of a suitable halotolerant and/or halophilic nuclease may be Saltonase, HL-Nuclease (EN32) available from Blirt®. If desired, the viral vector eluate may be diluted prior to nucleic acid cleavage. In some examples, the viral vector eluate may be subjected to buffer exchange prior to nucleic acid cleavage.

It has been found that the activity of certain nucleases such as Benzonase® Nuclease can be inhibited by, for instance, high salt conditions. For example, the highly concentrated salt buffers that may be used to elute bound viral vector from the anion exchanger may have a negative impact on the activity of endonucleases like Benzonase® Nuclease. To reduce the risk of this negative impact on activity, the highly concentrated salt may be exchanged from the eluate prior or during e.g. Benzonase® Nuclease treatment. For example, the eluate may be treated by buffer exchange prior or during e.g. Benzonase® Nuclease treatment to reduce the risk of the Benzonase® Nuclease being exposed to prolonged or excessive high salt conditions that may compromise nuclease activity.

Preferably, however, a halotolerant and/or halophilic nuclease (e.g. endonuclease) is used for nucleic acid cleavage. Halotolerant and/or halophilic nucleases are relatively active under high salt concentrations. In other words, their activity may be less negatively affected by high salt conditions than, for example, Benzonase®. Accordingly, it may be possible to treat the eluate from the anion exchanger using such nucleases without pre-treating the eluate by a salt-removal step. This can reduce the complexity of the process, improving process efficiency. Furthermore, high salt conditions can, at least in certain cases, promote dissociation of DNA from DNA-containing agglomerates or agglutinates. This can result in reduced agglomeration, which may be beneficial for the efficacy of downstream filtration steps. Furthermore, the unbound DNA may be more accessible to cleavage the halotolerant and/or halophilic nuclease. This can improve the efficacy of DNA removal. As mentioned above, an example of a suitable halotolerant and/or halophilic nuclease may be Salt Active Nuclease supplied by ArticZymes®. Another example of a suitable halotolerant and/or halophilic nuclease may be Saltonase, HL-Nuclease (EN32) available from Blirt®.

Following anion exchange, filtration may be performed. For example, the viral vector eluate from the anion exchanger may be filtered. Filtration may be performed after nucleic acid cleavage. For example, in one embodiment, once the viral vector eluate is treated with salt active nuclease, the viral vector eluate is filtered.

Where filtration is employed, any suitable filtration method may be used. Examples include tangential flow filtration.

### Ion-Exchange Chromatography

In ion-exchange chromatography, charged species, e.g. biomolecules, can bind reversibly to a stationary phase having groups of opposite charge. There are two types of ion exchangers: anion exchangers and cation exchangers. Anion exchangers are stationary phases that bear groups having a positive charge and hence can bind species with a negative charge. Cation exchangers bear groups with a negative charge and hence can bind species with positive charge. The pH of the medium can have an important influence on this, as it can alter the charge on a species.

Displacement (elution) of the bound species can be effected by the use of buffers.

The ionic concentration of the buffer may be increased until the species is displaced through competition of buffer ions for the ionic sites on the stationary phase. An alternative method of elution entails changing the pH of the buffer until the net charge of the species no longer favours binding to the stationary phase.

### Cation Exchanger

In the present disclosure, a viral vector preparation is passed through a cation exchanger. The flow-through from the cation exchanger is contacted with an anion exchanger that binds the viral vector, and the bound viral vector is then eluted from the anion exchanger as a viral vector eluate.

Any suitable cation exchanger may be employed. The cation exchanger may have a porous structure. For example, the cation exchanger may have a pore size of at least 1 μm, preferably at least 2 μm, more preferably at least 3 μm. The cation exchanger may be an acidic cation exchanger, for example, a strong acid or weak acid cation exchanger. The cation exchanger may comprise sulfonic acid ligands and/or carboxylic acid ligands, preferably sulfonic acid ligands. In some examples, the ligands may be covalently bound to internal surface of the cation exchanger. For example, the ligands may be present in the internal surface of the pores of the cation exchanger to provide a large surface area for binding.

In some examples, the cation exchanger may be a membrane. The membrane may have a porous structure. Acid ligands, for example, carboxylic acid and/or sulfonic acid ligands may be bound to the internal pore structure of the membrane. In some examples, the cation exchanger may be cation exchanger sold under the Sartobind S® trademark.

Where a cation exchange membrane is used, the cation exchange membrane may be treated with a suitable buffer to, for example, hydrate the membrane and/or flush out any leachable components that may be bound to the membrane. Any suitable chromatography buffer may be used in this step.

The membrane may then be sanitized, for example, with a suitable alkali solution. The membrane may then be flushed again with a suitable buffer. Any suitable chromatography buffer may be used in this step.

A high salt buffer may then be passed across the membrane to “charge” the functional groups on the cation exchange membrane. The membrane may then be equilibrated with a suitable chromatography buffer.

Following equilibration, the viral vector preparation may be contacted with the membrane. As explained further below, the viral preparation may be clarified supernatant from a viral vector cell culture.

As discussed above, DNA may interact or form complexes with e.g. positively charged species in the viral vector preparation, including, for example, histones. This interaction and/or complex-formation may allow DNA or DNA-containing complexes to bind to the cation exchanger, facilitating the separation of at least some DNA from the viral vector preparation by cation exchange. The flow-through treated by anion exchange, therefore, may contain reduced levels of DNA.

By passing a viral vector preparation through a cation exchanger, the risk of particle aggregation may also be reduced. As discussed above, excessive particle aggregation can be detrimental to the throughput of any downstream filtration steps, for example, by prematurely restricting or blocking the pores of filtration membranes. Although anion exchange can facilitate the separation of some aggregating histones because of the histones' positive charge, it has been found that, by carrying out cation exchange prior to the anion exchange step, improved removal of aggregating species, such as histones, from the viral vector preparation can be achieved.

Histones are a family of proteins that associate with DNA in the nucleus and help condense it into chromatin. Histones are basic proteins, and their positive charges allow them to associate with DNA, which is negatively charged. Some histones function as spools for the thread-like DNA to wrap around (core histones). There are five families of histones which are designated H1/H5 (linker histones), H2 (including H2A and H2B), H3, and H4 (core histones). Two of each of the four core histones H2A, H2B, H3 and H4 form a histone octamer around which approximately 146 bp of DNA are wrapped to form a nucleosomal core particle. The fifth histone, H1 binds to these nucleosomal core particles close to the DNA entry and exit sites and protects the free linker DNA (˜20 bp) between the individual nucleosomal core particles. These full nucleosomes including histone H1 (or isoform H5) may be referred to as chromatosomes. Full nucleosomes, in turn, can be wrapped into fibres that form chromatin.

Histones may bind to the cation exchanger during cation exchange because of their positive charge. This may allow separation of the histones from the flow-through. The manner in which histones bind may vary. For example, histones may bind as individual proteins. Alternatively or additionally, in some instances, histones may form aggregate structures with other histones (e.g. dimers, trimers, tetramers, pentamers, hexamers, heptamers, octamers or aggregates comprising combinations thereof) or other species (e.g. aggregates with DNA or other proteins). These aggregate structures may have surface positive charges that facilitate their separation by cation exchange.

As described above, the viral vector preparation may include at least one histone that is separated by cation exchange. Some histones, for example, the core histones such as H2 (including H2A and/or H2B), H3 and H4, preferably H2A, H2B and H3, may be suitably separated by cation exchange. In some instances, linker histones, for example, H1 or H5 may also be separated by cation exchange.

Where the histone is a H1 histone, the histone may be any member of the H1 histone family or any histone H1 subtype. For example the H1 histone may be, a somatic replication dependent subtype such as H1.1, H1.2, H1.3, H1.4 and/or H1.5, a somatic replication independent variant such as H1.0 and/or H1.10, a germ line-specific (testis or oocyte) subtype such as H1.6 and/or H1.7, and/or a splice variant of a germ line-specific subtype such as H1.8 and/or H1.9.

Where the histone is an H2 histone, the histone may be an H2A and/or H2B histone. Suitable H2A histones include H2AF, H2A1 and H2A2 histones.

Examples of H2AF histones include histones encoded by the:
H2AFB1 gene, such as H2A.B variant histone 1;
H2AFB2 gene such as H2A.B variant histone 2;
H2AFB3 gene such as H2A.B variant histone;
H2AFJ gene such as H2A.J histone;
H2AFV gene such as H2A.Z variant histone 2;
H2AFX gene such as H2A.X variant histone;
H2AFY gene such as core histone macro-H2A.1 (also known as MACROH2A1, H2A/y, H2A/y, H2AF12M, H2AFJ, MACROH2A1.1, mH2A1, macroH2A1.2, H2A histone family member Y, H2AFY, or macroH2A.1 histone);
H2AFY2 gene such as core histone macro-H2A.2 (also known as MACROH2A2, macroH2A2, H2A histone family member Y2, H2AFY2, macroH2A.2 histone); and/or
H2AFZ gene such as H2A.Z.

Examples of H2A1 histones include histones encoded by the:
HIST1H2AA gene such as Histone H2A type 1-A;
HIST1H2AB gene such as Histone H2A type 1-B;
HIST1H2AC gene such as Histone H2A type 1-C;
HIST1H2AD gene such as Histone H2A type 1-D;
HIST1H2AE, gene such as Histone H2A type 1-E;
HIST1H2AG, gene such as Histone H2A type 1-G;
HIST1H2AI, gene such as Histone H2A type 1-I;
HIST1H2AJ, gene such as Histone H2A type 1-J;
HIST1H2AK, gene such as Histone H2A type 1-K;
HIST1H2AL gene such as Histone H2A type 1-L; and
HIST1H2AM gene such as Histone H2A type 1-M.

Examples of H2A2 histones include histones encoded by the:
HIST2H2AA3 gene such as Histone H2A type 2-A;
HIST2H2AC gene such as Histone H2A type 2-C; and
HIST2H2AB gene such as Histone H2A type 2-B.

Suitable H2B histones include H2BF, H2B1 and H2B2 histones.

Examples of H2BF histones include histones encoded by the
H2BFM gene such as H2B histone family, member M;
H2BFS gene such as Histone H2B type F-S; and
H2BFWT gene such as H2B histone family, member W, testis-specific

Examples of H2B1 histones include histones encoded by the:
HIST1H2BA gene such as Histone H2B type 1-A;
HIST1H2BB gene such as Histone H2B type 1-B;
HIST1H2BC gene such as Histone H2B type 1-C;
HIST1H2BD gene such as Histone H2B type 1-D;
HIST1H2BE gene such as Histone H2B type 1-E;
HIST1H2BF gene such as Histone H2B type 1-F;
HIST1H2BG gene such as Histone H2B type 1-G;
HIST1H2BH gene such as Histone H2B type 1-H;
HIST1H2BI gene such as Histone H2B type 1-I;
HIST1H2BJ gene such as Histone H2B type 1-J;
HIST1H2BK gene such as Histone H2B type 1-K;
HIST1H2BL gene such as Histone H2B type 1-L;
HIST1H2BM gene such as Histone H2B type 1-M;
HIST1H2BN gene such as Histone H2B type 1-N; and
HIST1H2BO gene such as Histone H2B type 1-0.

Examples of H2B2 histones include histones encoded by the HIST2H2BE gene such as Histone H2B type 2-E.

Where the histone is an H3 histone, the histone may be an H3A1, H3A2 or H3A3 histone.

Examples of suitable H3A1 histones (also refereed to Histone H3.1) include histones encoded by the genes: HIST1H3A, HIST1H3B, HIST1H3C, HIST1H3D, HIST1H3E, HIST1H3F, HIST1H3G, HIST1H3H, HIST1H31, or HIST1H3J.

Examples of suitable H3A2 histones include histones encoded by the gene HIST2H3C such as Histone H3.2. An example of a suitable H3A3 histone is a histone encoded by the gene HIST3H3 such as H3.4 histone.

Where the histone is an H4 histone, the histone may be an H41 or H44 histone. Examples of H41 histones include histones encoded by the genes: HIST1H4A, HIST1H4B, HIST1H4C, HIST1H4D, HIST1H4E, HIST1H4F, HIST1H4G, HIST1H4H, HIST1H41, HIST1H4J, HIST1H4K or HIST1H4L.

An example of an H44 histone is a histone encoded by the gene HIST4H4.

Preferably, at least one of the following histones are separated by cation exchange: H4, H2 (e.g. H2B, H2A) and/or H3. Preferably, the histones separated by cation exchange are one or more of: core histone macro-H2A.1, core histone macro-H2A.2, Histone H2A type 2-C, Histone H2A type 2-B, Histone H2A.Z, Histone H2B type 1-K, at least one Histone H4, and/or at least one Histone H3.1.

The histones separated by cation exchange may have DNA bound thereto. As such removal of histones may also lead to removal of DNA bound thereto. Therefore, the use of cation exchange may reduce the amount of DNA in the viral vector preparation via binding and separation of histones from the viral vector preparation.

In particular, core histone proteins may be removed by cation exchange. Without being bound by theory, this may be due to the core histones function in binding negatively charged DNA to their surface, thus providing core histones with a positive surface charge which in turn leads to binding of the cation exchanger.

The use of cation exchange may lead to at least a 5-fold, preferably at least a 10-fold reduction in the amount of histones present in the viral vector preparation in comparison to the use of anion exchange alone. For example, the use of cation exchange may lead to at least a 10-fold reduction of H4, H2 (e.g. H2B, H2A) and/or H3 in comparison to the use of anion exchange alone. The use of cation exchange may lead to at least a 10-fold reduction in one or more of: core histone macro-H2A.1, core histone macro-H2A.2, Histone H2A type 2-C, Histone H2A type 2-B, Histone H2A.Z, Histone H2B type 1-K, at least one Histone H4, and/or at least one Histone H3 in comparison to the use of anion exchange alone.

The use of cation exchange may also lead to a reduction in other non-viral vector proteins present in viral vector preparation. Other proteins that may be bound by the cation exchanger may include histone binding proteins, ribosomal proteins, DNA binding proteins and/or RNA binding proteins. Similar to histones, these proteins may have a positive surface charge thus allowing them to be bound by the cation exchanger and separated from the viral vector preparation.

Examples of histone binding proteins that may be bound by the cation exchanger include histone methyltransferases, histone acetyltransferases, histone-binding protein RBBP4, histone-binding protein RBBP7, and histone deacetylases.

Examples of ribosomal proteins include small subunit ribosomal proteins and large subunit ribosomal proteins.

“DNA-binding proteins” refers to proteins that bind to DNA, including gene regulatory proteins, enzymes involved in DNA replication, recombination, repair, transcription, and degradation, and proteins involved in maintaining chromosome structure. They can be divided into two large groups: (1) Those that have some sequence- specific or secondary structure-specific requirement for DNA-binding, and (2) those that bind DNA nonspecifically. Examples of sequence-specific DNA-binding include homeodomain proteins; proteins involved in protein-nucleic acid interactions during recombination; restriction enzymes; and transcription factors. Examples of sequence- nonspecific DNA-binding include chromatin; proteins involved in DNA repair and DNA replication; and nucleases.

“RNA binding proteins” refers to RNA binding proteins involved in splicing and translation regulation such as tRNA binding proteins, RNA helicases, double-stranded RNA and single-stranded RNA binding proteins, mRNA binding proteins, snRNA cap binding proteins, 5S RNA and 7S RNA binding proteins, poly-pyrimidine tract binding proteins, snRNA binding proteins, and AU-specific RNA binding proteins.

As described above with respect to histones, histone binding proteins, ribosomal proteins, DNA binding proteins and/or RNA binding proteins may be bound to host cell DNA or RNA (e.g. in complexes with DNA or RNA) and as such binding of histone binding proteins, ribosomal proteins, DNA binding protein and/or RNA binding proteins may also lead to a reduction of host cell DNA in the viral vector preparation.

### Anion Exchanger

Once the viral vector preparation is contacted with the cation exchanger, the flow-through from the cation exchanger is contacted with an anion exchanger that binds the viral vector.

Any suitable anion exchange resin may be employed. The anion exchanger may have a porous structure. For example, the anion exchanger may have a pore size of at least 1 μm, preferably at least 2 μm, more preferably at least 3 μm. The anion exchanger may be a basic anion exchanger, for example, a strong base or weak base anion exchanger. The anion exchanger may include quaternary ammonium ligands. In some examples, the quaternary ammonium ligands may be covalently bound to internal surface of the anion exchanger. For example, the quaternary ammonium ligands may be present in the internal surface of the pores of the cation anion to provide a large surface area for binding.

In some examples, the anion exchanger may be a membrane. The membrane may have a porous structure. Quaternary ammonium ligands may be bound to the internal pore structure of the membrane. In some examples, the anion exchanger may be anion exchanger sold under the Sartobind Q® trademark.

Where an anion exchange membrane is used, it may be treated with a suitable chromatography buffer to e.g. rehydrate the membrane prior to use. The membrane may then be sanitized, for example, by treatment with sodium hydroxide, and then re-flushed with a suitable chromatography buffer.

The membrane may be charged by contacting the membrane with a high salt buffer.

The membrane may then be washed with a suitable chromatography buffer, before the flow through from the cation exchanger is contacted with the membrane. Any viral vector present in the flow-through may bind to the anion exchange sites.

To elute the bound viral vector, a suitable buffer, for example, a high salt buffer may be contacted with the anion exchanger. In some examples, the eluted vector sample may be diluted, for example, with a low-salt or salt-free buffer. This can reduce the exposure of vector to salt conditions, which may be detrimental to the vector.

### Nucleic Acid Cleavage

Once bound viral vector is eluted from the anion exchanger, the viral vector eluate may be subjected to nucleic acid cleavage. Nucleic acid cleavage may be employed to degrade, for example, any eluted DNA (e.g. originating from the host cell). Any suitable method of nucleic acid cleavage may be employed. For example, endonucleases or exonucleases may be used. Endonucleases are preferred.

Preferably, a halotolerant and/or halophilic nuclease (e.g. endonuclease) is used for nucleic acid cleavage. Halotolerant and/or halophilic nucleases are relatively active under high salt concentrations. In other words, their activity may be less negatively affected by high salt conditions than, for example, Benzonase®. Accordingly, it may be possible to treat the eluate from the anion exchanger using such nucleases without pre-treating the eluate by a salt-removal step. This can reduce the complexity of the process, improving process efficiency. Furthermore, high salt conditions can, at least in certain cases, promote dissociation of DNA from DNA-containing agglomerates or agglutinates. This can result in reduced agglomeration, which may be beneficial for the efficacy of downstream filtration steps. Furthermore, the unbound DNA may be more accessible to cleavage the halotolerant and/or halophilic nuclease. This can improve the efficacy of DNA removal. As mentioned above, an example of a suitable halotolerant and/or halophilic nuclease may be Salt Active Nuclease supplied by ArticZymes®. Another example of a suitable halotolerant and/or halophilic nuclease may be Saltonase, HL-Nuclease (EN32) available from Blirt®.

The nuclease may be suitable for degrading DNA and RNA (single stranded, double stranded linear or circular). The nuclease may hydrolyze nucleic acids by hydrolyzing internal phosphodiester bonds between specific nucleotides, thereby reducing the size of the polynucleotides in the vector containing supernatant. The concentration in which the nuclease is employed is preferably within the range of 1-100 units/ml.

Nucleic acid cleavage may be performed by contacting the sample with the nuclease for a suitable residence time and temperature. Nucleic acid cleavage may be performed within a hollow fibre membrane, whereby cleaved DNA may be removed through the membrane pores. Suitable residence times may range from 10 minutes to 5 hours, for example, 30 minutes to 3 hours, depending on the size of the sample and reaction conditions. Suitable reaction temperatures may range from 15 to 60 degrees C., for example, 30 to 50 degrees C. or 35 to 40 degrees C. (e.g. 37 degrees C.).

In some examples, the sample may be contacted with the nuclease in the presence of a co-factor. An example of a suitable co-factor is magnesium chloride. The nuclease may be employed in the presence of a co-factor, for example, magnesium chloride. In some examples, the nuclease may be Benzonase @Nuclease or a halotolerant and/or halophilic nuclease, such as Salt Active Nuclease. Where Benzonase® and/or Salt Active Nuclease is used, a co-factor such as magnesium chloride may also be present.

As discussed above, it has been found that the activity of certain nucleases such as Benzonase® Nuclease can be inhibited by, for instance, high salt conditions. For example, the salt buffers that may be used to elute bound viral vector from the anion exchanger may have a negative impact on the activity of endonucleases like Benzonase® Nuclease. To reduce the risk of this negative impact on activity, salt may be removed from the eluate prior or during e.g. Benzonase® Nuclease treatment. For example, the eluate may be treated by buffer exchange prior during e.g. Benzonase® Nuclease treatment to shield the Benzonase® Nuclease from prolonged or excessive exposure to high salt conditions.

Salt removal may be carried out by any suitable method. For example, the eluate from the anion exchanger may be treated by ultrafiltration to concentrate the viral vector. Buffer exchange into a suitable buffer for Benzonase® Nuclease treatment may then be performed, for example, by diafiltration. Nucleic acid treatment may be performed within the hollow fibre membranes.

Preferably, halotolerant and/or halophilic nuclease (e.g. endonuclease) is used for nucleic acid cleavage. Such nucleases are active under high salt concentrations. Accordingly, it may be possible to treat the eluate from the anion exchanger using such nucleases without pre-treating the eluate by a salt-removal step. This can reduce the complexity of the process, improving process efficiency. Furthermore, high salt conditions can, at least in certain cases, promote dissociation of DNA from DNA agglomerates. This can result in reduced agglomeration, which may be beneficial for the efficacy of downstream filtration steps. Furthermore, the unbound DNA may be more accessible to cleavage by Salt Active Nuclease. This can improve the efficacy of DNA removal. As mentioned above, an example of a suitable halotolerant and/or halophilic nuclease may be Salt Active Nuclease supplied by ArticZymes®. Another example of a suitable halotolerant and/or halophilic nuclease may be Saltonase, HL-Nuclease (EN32) available from Blirt®.

In addition to nucleic acid cleavage, selective precipitation (removal) of impurity DNA can be applied, e.g. by precipitation with an appropriate amount of a selective precipitation agent such as domiphen bromide (DB), CTAB (cetyl trimethylammonium bromide), cetylpyridinium chloride (CPC), benzethonium chloride (BTC), tetradecyltrimethyl- ammonium chloride (TTA), polyethylene imine (PEI), etc., as disclosed in detail in WO 03/097797.

### Filtration

The eluate from the anion exchanger may be subjected to filtration, for example, to concentrate the vector. Preferably, the eluate may be subjected to filtration once the eluate is treated by nucleic acid cleavage. Nucleic acid cleavage and filtration may be carried out within a hollow membrane structure.

With respect to filtration, any suitable filtration method may be employed. For example, the vector may be concentrated by passing the vector-containing feed through a filter membrane so that the vector is retained by the filter as a retentate.

An example of a suitable filtration method may be ultrafiltration. Ultrafiltration is a variety of membrane filtration in which forces like pressure or concentration gradients lead to a separation through a semipermeable membrane. Suspended solids and solutes of high molecular weight are retained in the so-called retentate, while water and low molecular weight solutes pass through the membrane in the permeate (filtrate). This separation process may be used for purifying and concentrating macromolecular (10³-10⁶ Da) solutions.

In some examples, the filter membrane selected for filtration may have a pore size sufficiently small to retain vector but large enough to effectively allow some impurities to pass therethrough. In certain embodiments, hollow fibers of 500 kDa (0.05 μm) pore size are used according to examples of the present disclosure.

The membrane composition may be, but is not limited to, regenerated cellulose, polyethersulfone, polysulfone, or derivatives thereof.

The membranes can be flat sheets (also called flat screens), tubular modules, hollow fibers and/or spiral-wound modules. A preferred membrane is hollow fibre membrane.

In some instances, it may be desirable to perform buffer exchange. For example, following nucleic acid cleavage, the viral vector sample may be filtered and its buffer exchanged for a buffer more suitable for storage. For instance, after nucleic acid cleavage is performed using Salt Active Nuclease or Benzonase @nuclease, the viral vector sample may be filtered and the buffer exchanged for a buffer that is suitable for storage of the vector. As an example, a buffer solution comprising Tromethamine may be used.

Where nucleic acid cleavage is performed using a nuclease (e.g. Benzonase® nuclease) that is sensitive e.g. to high salt buffers used to elute the viral vector from the anion exchanger, it may be desirable to perform a buffer exchange prior to nucleic acid cleavage. In this instance, the e.g. high salt buffer from the anion exchanger may be exchanged with a buffer having a lower salt content. As an example, a buffer solution comprising Tromethamine may be used. With nucleases e.g. Benzonase® nuclease, buffer exchange may be performed before and after nucleic acid cleavage.

Buffer exchange may be performed using any suitable method. Preferably, buffer exchange may be performed by diafiltration. Diafiltration may involve the addition of buffer during filtration, such that the buffer solution surrounding the target viral vector is changed. This rebuffering step may stabilize the viral vector, for example, for storage. In some examples, filtration may be carried out to pre-concentrate the viral vector, prior to re-buffering by diafiltration.

Filtration may be carried out using any suitable set-up. For example, filtration may be performed by direct flow filtration in which feed is passed through the filter membrane, whereby solids are trapped by the filter and the filtrate or permeate passes through the membrane. Alternatively, filtration may be performed by tangential flow filtration. In tangential flow filtration, the feed is passed across the filter membrane (tangentially) at positive pressure relative to the permeate side. A proportion of the material which is smaller than the membrane pore size passes through the membrane as permeate or filtrate, while the remainder is retained on the feed side of the membrane as retentate. An advantage of this is that the residue that can blind the filter may be substantially washed away during the filtration process, increasing the length of time that a filter unit can be operational. Tangential flow filtration can be a continuous process.

Following filtration, the viral vector may be frozen for storage. Suitable freezing temperatures include temperatures of −40 degrees C. or lower, for example, −60 degrees C. or lower e.g. −80 degrees C.

Following freezing, the viral vector may be thawed, subjected to sterile filtration and concentrated (e.g. by ultrafiltration) before being placed in vials for further storage e.g. at low temperatures.

### Viral Vector Preparation

As discussed above, the viral vector preparation may be produced using any suitable method. Preferably, the viral vector preparation is produced by culturing cells that produce the viral vector and harvesting a viral vector-containing supernatant from the cell culture.

General methods for producing viral vector from a cell (producer/production cell) comprising nucleic acid sequences encoding viral vector components are well known in the art. These methods comprise culturing the cell under conditions suitable for the production of the viral vectors.

A viral vector production systems may be used for producing viral vector preparations as described herein. Viral vector production systems comprise a set of nucleotide sequences encoding the components required for production of the viral vector. Accordingly, a vector production system comprises a set of nucleotide sequences which encode the components necessary to generate viral vector particles. Typically, the set of nucleotide sequences is present within a cell.

“Viral vector production system” or “vector production system” or “production system” is to be understood as a system comprising the necessary components for viral vector production. The terms “components required for production of the vector” and “viral vector components” are used interchangeably herein. The viral vector production system comprises a set of nucleotide sequences which encode the components necessary to generate viral vector particles.

In the context of the methods described herein, the viral vectors may be retroviral vectors. A viral vector may also be called a vector, vector virion or vector particle. The vectors may contain one or more selectable marker genes (e.g. a neomycin resistance gene) and/or traceable marker gene(s) (e.g. a gene encoding green fluorescent protein (GFP)). Vectors may be used, for example, to infect and/or transduce a target cell.

A non-limiting example of a viral vector production system described herein is a lentiviral vector production system. A lentiviral vector production system comprises a set of nucleotide sequences encoding the components required for production of a lentiviral vector. A lentiviral vector production system therefore comprises a set of nucleotide sequences which encode the components necessary to generate lentiviral vector particles. As stated above, the set of nucleotide sequences is typically present within a cell.

In one example, the set of nucleotide sequences may be suitable for generation of a lentiviral vector in a tat-independent system for vector production. 3^{rd} generation lentiviral vectors are U3-dependent (and employ a heterologous promoter to drive transcription). In one example, tat is not provided in the lentiviral vector production system, for example tat is not provided in trans.

The set of nucleotide sequences may comprise nucleotide sequences encoding Gag and Gag/Pol proteins, and Env protein and the viral vector genome sequence. The set of nucleotide sequences may optionally comprise a nucleotide sequence encoding the Rev protein, or functional substitute thereof.

The viral vector production system may comprise modular nucleic acid constructs (modular constructs). A modular construct is a DNA expression construct comprising two or more nucleic acids used in the production of viral vectors. A modular construct can be a DNA plasmid comprising two or more nucleic acids used in the production of viral vectors. The plasmid may be a bacterial plasmid. The nucleic acids can encode for example, gag-pol, rev, env, and/or viral vector genome. In addition, modular constructs designed for generation of packaging and producer cell lines may additionally need to encode transcriptional regulatory proteins (e.g. TetR, CymR) and/or translational repression proteins (e.g. TRAP) and selectable markers (e.g. Zeocin™, hygromycin, blasticidin, puromycin, neomycin resistance genes). Suitable modular constructs are described in EP 3502260, which is hereby incorporated by reference in its entirety.

As modular constructs contain nucleic acid sequences encoding two or more of the viral components on one construct, the safety profile of these modular constructs has been considered and additional safety features directly engineered into the constructs. These features include the use of insulators for multiple open reading frames of viral vector components and/or the specific orientation and arrangement of the viral genes in the modular constructs. It is believed that by using these features the direct read-through to generate replication-competent viral particles will be prevented.

The nucleic acid sequences encoding the viral vector components may be in reverse and/or alternating transcriptional orientations in the modular construct. Thus, the nucleic acid sequences encoding the viral vector components are not presented in the same 5′ to 3′ orientation, such that the viral vector components cannot be produced from the same mRNA molecule. The reverse orientation may mean that at least two coding sequences for different vector components are presented in the ‘head-to-head’ and ‘tail-to-tail’ transcriptional orientations. This may be achieved by providing the coding sequence for one vector component, e.g. env, on one strand and the coding sequence for another vector component, e.g. rev, on the opposing strand of the modular construct. Preferably, when coding sequences for more than two vector components are present in the modular construct, at least two of the coding sequences are present in the reverse transcriptional orientation. Accordingly, when coding sequences for more than two vector components are present in the modular construct, each component may be orientated such that it is present in the opposite 5′ to 3′ orientation to all of the adjacent coding sequence(s) for other vector components to which it is adjacent, i.e. alternating 5′ to 3′ (or transcriptional) orientations for each coding sequence may be employed.

The modular construct may comprise nucleic acid sequences encoding two or more of the following vector components: gag-pol, rev, env, vector genome. The modular construct may comprise nucleic acid sequences encoding any combination of the vector components. In one example, the modular construct may comprise nucleic acid sequences encoding:
i) the RNA genome of a retroviral vector and rev, or a functional substitute thereof;
ii) the RNA genome of a retroviral vector and gag-pol;
iii) the RNA genome of a retroviral vector and env;
iv) gag-pol and rev, or a functional substitute thereof;
v) gag-pol and env;
vi) env and rev, or a functional substitute thereof;
vii) the RNA genome of a retroviral vector, rev, or a functional substitute thereof, and gag-pol;
viii) the RNA genome of a retroviral vector, rev, or a functional substitute thereof, and env;
ix) the RNA genome of a retroviral vector, gag-pol and env; or
x) gag-pol, rev, or a functional substitute thereof, and env,
wherein the nucleic acid sequences are in reverse and/or alternating orientations.

In some examples, the retroviral vector may be a lentiviral vector.

As stated elsewhere herein, the viral vector production system described herein typically comprises the nucleic acid sequences encoding viral vector components within a cell (in other words, a cell comprises the nucleic acid sequences encoding viral vector components). In one example, the cell of the viral vector production system may comprise nucleic acid sequences encoding any one of the combinations i) to x) above, wherein the nucleic acid sequences are located at the same genetic locus and are in reverse and/or alternating orientations. The same genetic locus may refer to a single extrachromosomal locus in the cell, e.g. a single plasmid, or a single locus (i.e. a single insertion site) in the genome of the cell. The cell may be a stable or transient cell for producing retroviral vectors, e.g. lentiviral vectors.

The DNA expression construct can be a DNA plasmid (supercoiled, nicked or linearized), minicircle DNA (linear or supercoiled), plasmid DNA containing just the regions of interest by removal of the plasmid backbone by restriction enzyme digestion and purification, DNA generated using an enzymatic DNA amplification platform e.g. doggybone DNA (dbDNA™) where the final DNA used is in a closed ligated form or where it has been prepared (e.g. restriction enzyme digestion) to have open cut ends.

A “viral vector production cell”, “vector production cell”, or “production cell” is to be understood as a cell that is capable of producing a viral vector or viral vector particle. Viral vector production cells may be “producer cells” or “packaging cells”. One or more DNA constructs of the viral vector system may be either stably integrated or episomally maintained within the viral vector production cell. Alternatively, all the DNA components of the viral vector system may be transiently transfected into the viral vector production cell. In yet another alternative, a production cell stably expressing some of the components may be transiently transfected with the remaining components required for vector production.

As used herein, the term “packaging cell” refers to a cell which contains the elements necessary for production of viral vector particles but which lacks the vector genome. Optionally, such packaging cells contain one or more expression cassettes which are capable of expressing viral structural proteins (such as gag, gag/pol and env).

Producer cells/packaging cells can be of any suitable cell type. Producer cells are generally mammalian cells but can be, for example, insect cell.

As used herein, the term “producer/production cell” or “vector producing/production cell” refers to a cell which contains all the elements necessary for production of viral vector particles. The producer cell may be either a stable producer cell line or derived transiently or may be a stable packaging cell wherein the viral genome is transiently expressed.

The vector production cells may be cells cultured in vitro such as a tissue culture cell line. Suitable production cells or cells for producing a viral vector may be cells which are capable of producing viral vectors or viral vector particles when cultured under appropriate conditions. Thus, the cells typically comprise nucleotide sequences encoding vector components, which may include gag, env, rev and the genome of the viral vector. Suitable cell lines include, but are not limited to, mammalian cells such as murine fibroblast derived cell lines or human cell lines. They are generally mammalian, including human cells, for example HEK293T, HEK293, CAP, CAP-T or CHO cells, but can be, for example, insect cells such as SF9 cells. Preferably, the vector production cells are derived from a human cell line. Accordingly, such suitable production cells may be employed to provide a viral preparation as described herein for use in any of the methods of the present invention.

Methods for introducing nucleotide sequences into cells are well known in the art. Thus, the introduction into a cell of nucleotide sequences encoding vector components including gag, env, rev and the genome of the viral vector using conventional techniques in molecular and cell biology is within the capabilities of a person skilled in the art.

Stable production cells may be packaging or producer cells. To generate producer cells from packaging cells the vector genome DNA construct may be introduced stably or transiently. Packaging/producer cells can be generated by transducing a suitable cell line with a retroviral vector which expresses one of the components of the vector, i.e. a genome, the gag-pol components and an envelope as described in WO 2004/022761 which is incorporated herein by reference. Alternatively, the nucleotide sequence can be transfected into cells and then integration into the production cell genome occurs infrequently and randomly. The transfection methods may be performed using methods well known in the art. For example, a stable transfection process may employ constructs which have been engineered to aid concatemerisation. In another example, the transfection process may be performed using calcium phosphate or commercially available formulations such as Lipofectamine™ 2000CD (Invitrogen, CA), FuGENE@HD or polyethylenimine (PEI). Alternatively nucleotide sequences may be introduced into the production cell via electroporation. The skilled person will be aware of methods to encourage integration of the nucleotide sequences into production cells. For example, linearising a nucleic acid construct can help if it is naturally circular. Less random integration methodologies may involve the nucleic acid construct comprising of areas of shared homology with the endogenous chromosomes of the mammalian host cell to guide integration to a selected site within the endogenous genome. Furthermore, if recombination sites are present on the construct then these can be used for targeted recombination. For example, the nucleic acid construct may contain a loxP site which allows for targeted integration when combined with Cre recombinase (i.e. using the Cre/lox system derived from P1 bacteriophage). Alternatively or additionally, the recombination site is an att site (e.g. from A phage), wherein the att site permits site-directed integration in the presence of a lambda integrase. This would allow the viral genes to be targeted to a locus within the host cellular genome which allows for high and/or stable expression.

Other methods of targeted integration are well known in the art. For example, methods of inducing targeted cleavage of genomic DNA can be used to encourage targeted recombination at a selected chromosomal locus. These methods often involve the use of methods or systems to induce a double strand break (DSB) e.g. a nick in the endogenous genome to induce repair of the break by physiological mechanisms such as non-homologous end joining (NHEJ). Cleavage can occur through the use of specific nucleases such as engineered zinc finger nucleases (ZFN), transcription-activator like effector nucleases (TALENs), using CRISPR/Cas9 systems with an engineered crRNA/tracr RNA (‘single guide RNA’) to guide specific cleavage, and/or using nucleases based on the Argonaute system (e.g., from T. thermophilus).

Packaging/producer cell lines can be generated by integration of nucleotide sequences using methods of just viral transduction or just nucleic acid transfection, or a combination of both can be used.

Methods for generating retroviral vectors from production cells and in particular the processing of retroviral vectors are described in WO 2009/153563 which is incorporated herein by reference.

In one example, the production cell may comprise the RNA-binding protein (e.g. tryptophan RNA-binding attenuation protein, TRAP) and/or the Tet Repressor (TetR) protein or alternative regulatory proteins (e.g. CymR).

Production of viral vector from production cells can be via transfection methods, from production from stable cell lines which can include induction steps (e.g. doxycycline induction) or via a combination of both. The transfection methods may be performed using methods well known in the art, and examples have been described previously.

Production cells, either packaging or producer cell lines or those transiently transfected with the viral vector encoding components are cultured to increase cell and virus numbers and/or virus titres. Culturing a cell is performed to enable it to metabolize, and/or grow and/or divide and/or produce viral vectors of interest. This can be accomplished by methods well known to persons skilled in the art, and includes but is not limited to providing nutrients for the cell, for instance in the appropriate culture media. The methods may comprise growth adhering to surfaces, growth in suspension, or combinations thereof. Culturing can be done for instance in tissue culture flasks, tissue culture multiwell plates, dishes, roller bottles, wave bags or in bioreactors, using batch, fed-batch, continuous systems and the like. In order to achieve large scale production of viral vector through cell culture it is preferred in the art to have cells capable of growing in suspension. Suitable conditions for culturing cells are known (see e.g. Tissue Culture, Academic Press, Kruse and Paterson, editors (1973), and R.I. Freshney, Culture of animal cells: A manual of basic technique, fourth edition (Wiley- Liss Inc., 2000, ISBN 0-471-34889-9).

Cells may initially be ‘bulked up’ in tissue culture flasks or bioreactors and subsequently grown in multi-layered culture vessels or large bioreactors (greater than 50L) to generate the vector producing cells.

Cells may be grown in an adherent mode to generate the vector producing cells. Alternatively, cells may be grown in a suspension mode to generate the vector producing cells.

Once the cells have been grown the viral vector is harvested from the cells or culture medium (supernatant). In the case of harvesting from the culture medium, the culture medium may be removed from the flask or bioreactor containing the cells and includes the virus vector. In the case of harvesting from cells, the cells may be lysed. “Lysing” refers to disrupting the cellular membrane and optionally cell wall of a cell sufficient to release at least some intracellular contents, in this a viral vector. Methods of lysing cells are well known and include enzymatic lysis, chemical lysis and physical lysis methods.

Nucleic acid impurities can originate from either plasmid DNA transfection or host cell DNA from the producer cell lines. Nucleic acids may need to be reduced during processing to improve purity and reduce the risk of deleterious effect in recipient cells or patients. This may be achieved by the addition of nucleases, such as benzonase. In some cases, nucleases may be added directly to the viral vector culture e.g. prior to or after harvesting, or after clarification.

The degree to which positively charged contaminating protein (such as DNA binding proteins like histones) may remain bound to residual DNA within the viral vector harvest material may depend on the efficiency of treatment of the viral vector harvest with nucleases. Such nucleases may be supplied as commercially available recombinant enzymes such as Benzonase or SAN, or may be provided de novo by co-expression in the viral vector culture as described by the SecNuc system (WO2019/175600A1). In instances where efficient degradation of residual DNA within viral vector harvest material has been achieved prior to purification, the level of positively charged contaminating protein (such as DNA binding proteins like histones) can be expected to be high. Some of these positively charged proteins and/or protein complexes may become associated with negatively charged viral vector particles, hampering downstream purification. Therefore the use of a cation-exchange step prior to anion-exchange to remove such contaminants may be desirable when employing efficient nuclease-based treatments in viral vector harvest material.

### Lentiviral Vectors

As discussed above, the viral vector preparations treated herein may be lentiviral vector preparations.

Lentiviruses are part of a larger group of retroviruses. A detailed list of lentiviruses may be found in Coffin et al (1997) “Retroviruses” Cold Spring Harbour Laboratory Press Eds: J M Coffin, SM Hughes, HE Varmus pp 758-763). In brief, lentiviruses can be divided into primate and non-primate groups. Examples of primate lentiviruses include but are not limited to: the human immunodeficiency virus (HIV), the causative agent of human auto-immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV), feline immunodeficiency virus (FIV), Maedi visna virus (MVV) and bovine immunodeficiency virus (BIV). In one aspect, the lentiviral vector is derived from HIV-1, HIV-2, SIV, FIV, BIV, EIAV, CAEV or Visna lentivirus.

The lentivirus family differs from retroviruses in that lentiviruses have the capability to infect both dividing and non-dividing cells (Lewis et al (1992) EMBO J 11(8):3053-3058 and Lewis and Emerman (1994) J Virol 68 (1):510-516). In contrast, other retroviruses, such as MLV, are unable to infect non-dividing or slowly dividing cells such as those that make up, for example, muscle, brain, lung and liver tissue.

A lentiviral vector, as used herein, is a vector which comprises at least one component part derivable from a lentivirus. Preferably, that component part is involved in the biological mechanisms by which the vector infects or transduces target cells and expresses a nucleotide of interest (NOI), or nucleotides of interest.

The lentiviral vector may be used to replicate the NOI in a compatible target cell in vitro. Protein and NOI may be recovered from the target cell by methods well known in the art. Suitable target cells include mammalian cell lines and other eukaryotic cell lines.

In some aspects the vectors may have “insulators”—genetic sequences that block the interaction between promoters and enhancers, and act as a barrier reducing read-through from an adjacent gene.

In one aspect the insulator is present between one or more of the lentiviral nucleic acid sequences to prevent promoter interference and read-thorough from adjacent genes. If the insulators are present in the vector between one or more of the lentiviral nucleic acid sequences, then each of these insulated genes may be arranged as individual expression units.

The basic structure of retroviral and lentiviral genomes share many common features such as a 5′ LTR and a 3′ LTR, between or within which are located a packaging signal to enable the genome to be packaged, a primer binding site, integration sites to enable integration into a target cell genome and gag/pol and env genes encoding the packaging components—these are polypeptides required for the assembly of viral particles. Lentiviruses have additional features, such as the rev gene and RRE sequences in HIV, which enable the efficient export of RNA transcripts of the integrated provirus from the nucleus to the cytoplasm of an infected target cell.

In the provirus, these genes are flanked at both ends by regions called long terminal repeats (LTRs). The LTRs are responsible for proviral integration, and transcription. LTRs also serve as enhancer-promoter sequences and can control the expression of the viral genes.

The LTRs themselves are identical sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

In a typical retroviral vector, at least part of one or more protein coding regions essential for replication may be removed from the virus; for example, gag/pol and env may be absent or not functional. This makes the viral vector replication-defective.

The lentiviral vector may be derived from either a primate lentivirus (e.g. HIV-1) or a non-primate lentivirus (e.g. EIAV).

In general terms, a typical retroviral vector production system involves the separation of the viral genome from the essential viral packaging functions. These viral vector components are normally provided to the production cells on separate DNA expression cassettes (alternatively known as plasmids, expression plasmids, DNA constructs or expression constructs).

The vector genome comprises the NOI. Vector genomes typically require a packaging signal (Ψ), the internal expression cassette harbouring the NOI, (optionally) a post-transcriptional element (PRE), typically a central polypurine tract (cppt), the 3′-ppu and a self-inactivating (SIN) LTR. The R-U5 regions are required for correct polyadenylation of both the vector genome RNA and NOI mRNA, as well as the process of reverse transcription. The vector genome may optionally include an open reading frame, as described in WO 2003/064665, which allows for vector production in the absence of rev.

The packaging functions include the gag/pol and env genes. These are required for the production of vector particles by the production cell. Providing these functions in trans to the genome facilitates the production of replication-defective viral vectors.

Production systems for gamma-retroviral vectors are typically 3-component systems requiring genome, gag/pol and env expression constructs. Production systems for HIV-1-based lentiviral vectors may additionally require the accessory gene rev to be provided and for the vector genome to include the rev-responsive element (RRE). EIAV-based lentiviral vectors do not require rev to be provided in trans if an open-reading frame (ORF) is present within the genome (see WO 2003/064665).

Usually both the “external” promoter (which drives the vector genome cassette) and “internal” promoter (which drives the NOI cassette) encoded within the vector genome cassette are strong eukaryotic or virus promoters, as are those driving the other vector system components. Examples of such promoters include CMV, EF1α, PGK, CAG, TK, SV40 and Ubiquitin promoters. Strong ‘synthetic’ promoters, such as those generated by DNA libraries (e.g. JeT promoter) may also be used to drive transcription. Alternatively, tissue-specific promoters such as rhodopsin (Rho), rhodopsin kinase (RhoK), cone-rod homeobox containing gene (CRX), neural retina-specific leucine zipper protein (NRL), Vitelliform Macular Dystrophy 2 (VMD2), Tyrosine hydroxylase, neuronal-specific neuronal-specific enolase (NSE) promoter, astrocyte-specific glial fibrillary acidic protein (GFAP) promoter, human α1-antitrypsin (hAAT) promoter, phosphoenolpyruvate carboxykinase (PEPCK), liver fatty acid binding protein promoter, Flt-1 promoter, INF-β promoter, Mb promoter, SP-B promoter, SYN1 promoter, WASP promoter, SV40/hAlb promoter, SV40/CD43, SV40/CD45, NSE/RU5′ promoter, ICAM-2 promoter, GPIIb promoter, GFAP promoter, Fibronectin promoter, Endoglin promoter, Elastase-1 promoter, Desmin promoter, CD68 promoter, CD14 promoter and B29 promoter may be used to drive transcription.

Production of retroviral vectors involves either the transient co-transfection of the production cells with these DNA components or use of stable production cell lines wherein all the components are stably integrated within the production cell genome (e.g. Stewart H J, Fong-Wong L, Strickland I, Chipchase D, Kelleher M, Stevenson L, Thoree V, McCarthy J, Ralph G S, Mitrophanous K A and Radcliffe P A. (2011). Hum Gene Ther. March; 22 (3):357-69). An alternative approach is to use a stable packaging cell (into which the packaging components are stably integrated) and then transiently transfect in the vector genome plasmid as required (e.g. Stewart, H. J., M. A. Leroux-Carlucci, C. J. Sion, K. A. Mitrophanous and P. A. Radcliffe (2009). Gene Ther. June; 16 (6):805-14). It is also feasible that alternative, not complete, packaging cell lines could be generated (just one or two packaging components are stably integrated into the cell lines) and to generate vector the missing components are transiently transfected. The production cell may also express regulatory proteins such as a member of the tet repressor (TetR) protein group of transcription regulators (e.g.T-Rex, Tet-On, and Tet-Off), a member of the cumate inducible switch system group of transcription regulators (e.g. cumate repressor (CymR) protein), or an RNA-binding protein (e.g. TRAP-tryptophan-activated RNA-binding protein).

In one example, the viral vector is derived from EIAV. EIAV has the simplest genomic structure of the lentiviruses and is particularly preferred for use in the present invention. In addition to the gag/pol and env genes, EIAV encodes three other genes: tat, rev, and S2. Tat acts as a transcriptional activator of the viral LTR (Derse and Newbold (1993) Virology 194(2):530-536 and Maury et al (1994) Virology 200(2):632-642) and rev regulates and coordinates the expression of viral genes through rev-response elements (RRE) (Martarano et al. (1994) J Virol 68(5):3102-3111). The mechanisms of action of these two proteins are thought to be broadly similar to the analogous mechanisms in the primate viruses (Martarano et al. (1994) J Virol 68(5):3102-3111). The function of S2 is unknown. In addition, an EIAV protein, Ttm, has been identified that is encoded by the first exon of tat spliced to the env coding sequence at the start of the transmembrane protein. In an alternative aspect of the present invention the viral vector is derived from HIV: HIV differs from EIAV in that it does not encode S2 but unlike EIAV it encodes vif, vpr, vpu and nef.

The term “recombinant retroviral or lentiviral vector” (RRV) refers to a vector with sufficient retroviral genetic information to allow packaging of an RNA genome, in the presence of packaging components, into a viral particle capable of transducing a target cell. Transduction of the target cell may include reverse transcription and integration into the target cell genome. The RRV carries non-viral coding sequences which are to be delivered by the vector to the target cell. A RRV is incapable of independent replication to produce infectious retroviral particles within the target cell. Usually the RRV lacks a functional gag/pol and/or env gene, and/or other genes essential for replication.

Preferably the RRV vector has a minimal viral genome.

As used herein, the term “minimal viral genome” means that the viral vector has been manipulated so as to remove the non-essential elements whilst retaining the elements essential to provide the required functionality to infect, transduce and deliver a NOI to a target cell. Further details of this strategy can be found in WO 1998/17815 and WO 99/32646. A minimal EIAV vector lacks tat, rev and S2 genes and neither are these genes provided in trans in the production system. A minimal HIV vector lacks vif, vpr, vpu, tat and nef.

The expression plasmid used to produce the vector genome within a production cell may include transcriptional regulatory control sequences operably linked to the retroviral genome to direct transcription of the genome in a production cell/packaging cell. All 3rd generation lentiviral vectors are deleted in the 5′ U3 enhancer-promoter region, and transcription of the vector genome RNA is driven by heterologous promoter such as another viral promoter, for example the CMV promoter, as discussed below. This feature enables vector production independently of tat. Some lentiviral vector genomes require additional sequences for efficient virus production. For example, particularly in the case of HIV, RRE sequences may be included. However the requirement for RRE on the (separate) GagPol cassette (and dependence on rev which is provided in trans) may be reduced or eliminated by codon optimisation of the GagPol ORF. Further details of this strategy can be found in WO 2001/79518.

Alternative sequences which perform the same function as the rev/RRE system are also known. For example, a functional analogue of the rev/RRE system is found in the Mason Pfizer monkey virus. This is known as the constitutive transport element (CTE) and comprises an RRE-type sequence in the genome which is believed to interact with a factor in the infected cell. The cellular factor can be thought of as a rev analogue. Thus, CTE may be used as an alternative to the rev/RRE system. Any other functional equivalents of the Rev protein which are known or become available may be relevant to the invention. For example, it is also known that the Rex protein of HTLV-I can functionally replace the Rev protein of HIV-1. Rev and RRE may be absent or non-functional in the vector for use in the methods of the present invention; in the alternative rev and RRE, or functionally equivalent system, may be present.

It is therefore understood that ‘rev’ may refer to a sequence encoding the HIV-1 Rev protein or a sequence encoding any functional equivalent thereof. Thus, in an aspect, the invention provides a viral vector production system and/or a cell comprising a set of nucleotide sequences, wherein the nucleotide sequences encode vector components including gag-pol, env, optionally rev, and the nucleotide sequences of any of the preceding claims.

As used herein, the term “functional substitute” means a protein or sequence having an alternative sequence which performs the same function as another protein or sequence. The term “functional substitute” is used interchangeably with “functional equivalent” and “functional analogue” herein with the same meaning.

### SIN Vectors

The lentiviral vectors as described herein may be used in a self-inactivating (SIN) configuration in which the viral enhancer and promoter sequences have been deleted. SIN vectors can be generated and transduce non-dividing target cells in vivo, ex vivo or in vitro with an efficacy similar to that of non-SIN vectors. The transcriptional inactivation of the long terminal repeat (LTR) in the SIN provirus should prevent mobilisation of vRNA, and is a feature that further diminishes the likelihood of formation of replication-competent virus. This should also enable the regulated expression of genes from internal promoters by eliminating any cis-acting effects of the LTR.

By way of example, self-inactivating retroviral vector systems have been constructed by deleting the transcriptional enhancers or the enhancers and promoter in the U3 region of the 3′ LTR. After a round of vector reverse transcription and integration, these changes are copied into both the 5′ and the 3′ LTRs producing a transcriptionally inactive provirus. However, any promoter(s) internal to the LTRs in such vectors will still be transcriptionally active. This strategy has been employed to eliminate effects of the enhancers and promoters in the viral LTRs on transcription from internally placed genes. Such effects include increased transcription or suppression of transcription. This strategy can also be used to eliminate downstream transcription from the 3′ LTR into genomic DNA. This is of particular concern in human gene therapy where it is important to prevent the adventitious activation of any endogenous oncogene. Yu et al., (1986) PNAS 83: 3194-98; Marty et al., (1990) Biochimie 72: 885-7; Naviaux et al., (1996) J. Virol. 70: 5701-5; Iwakuma et al., (1999) Virol. 261: 120-32; Deglon et al., (2000) Human Gene Therapy 11: 179-90. SIN lentiviral vectors are described in U.S. Pat. Nos. 6,924,123 and 7,056,699.

### Replication-Defective Lentiviral Vectors

In the genome of a replication-defective lentiviral vector the sequences of gag/pol and/or env may be mutated and/or not functional.

In a typical lentiviral vector as described herein, at least part of one or more coding regions for proteins essential for virus replication may be removed from the vector. This makes the viral vector replication-defective. Portions of the viral genome may also be replaced by a NOI in order to generate a vector comprising an NOI which is capable of transducing a non-dividing target cell and/or integrating its genome into the target cell genome.

In one aspect the lentiviral vectors are non-integrating vectors as described in WO 2006/010834 and WO 2007/071994.

In a further aspect the vectors have the ability to deliver a sequence which is devoid of or lacking viral RNA. In a further aspect a heterologous binding domain (heterologous to gag) located on the RNA to be delivered and a cognate binding domain on Gag or GagPol can be used to ensure packaging of the RNA to be delivered. Both of these vectors are described in WO 2007/072056.

### Envelope and Pseudotyping

In one preferred aspect, the lentiviral vector has been pseudotyped. In this regard, pseudotyping can confer one or more advantages. For example, the env gene product of the HIV based vectors would restrict these vectors to infecting only cells that express a protein called CD4. But if the env gene in these vectors has been substituted with env sequences from other enveloped viruses, then they may have a broader infectious spectrum (Verma and Somia (1997) Nature 389(6648):239-242). By way of example, workers have pseudotyped an HIV based vector with the glycoprotein from VSV (Verma and Somia (1997) Nature 389(6648):239-242). Accordingly, alternative sequences which perform the equivalent function as the env gene product of HIV based vectors are also known.

In another alternative, the Env protein may be a modified Env protein such as a mutant or engineered Env protein. Modifications may be made or selected to introduce targeting ability or to reduce toxicity or for another purpose (Valsesia-Wittman et al 1996 J Virol 70: 2056-64; Nilson et al (1996) Gene Ther 3(4):280-286; and Fielding et al (1998) Blood 91(5):1802-1809 and references cited therein).

The vector may be pseudotyped with any molecule of choice.

As used herein, “env” shall mean an endogenous lentiviral envelope or a heterologous envelope, as described herein. Suitably, env may be Env of HIV based vectors or a functional substitute thereof.

### VSVG

The envelope glycoprotein (G) of Vesicular stomatitis virus (VSV), a rhabdovirus, is an envelope protein that has been shown to be capable of pseudotyping certain enveloped viruses and viral vector virions.

Its ability to pseudotype MoMLV-based retroviral vectors in the absence of any retroviral envelope proteins was first shown by Emi et al. (1991) Journal of Virology 65:1202-1207. WO 1994/294440 teaches that retroviral vectors may be successfully pseudotyped with VSV-G. These pseudotyped VSV-G vectors may be used to transduce a wide range of mammalian cells. More recently, Abe et al. (1998) J Virol 72(8) 6356-6361 teach that non-infectious retroviral particles can be made infectious by the addition of VSV-G.

Burns et al. (1993) Proc. Natl. Acad. Sci. USA 90:8033-7 successfully pseudotyped the retrovirus MLV with VSV-G and this resulted in a vector having an altered host range compared to MLV in its native form. VSV-G pseudotyped vectors have been shown to infect not only mammalian cells, but also cell lines derived from fish, reptiles and insects (Burns et al. (1993) ibid). They have also been shown to be more efficient than traditional amphotropic envelopes for a variety of cell lines (Yee et al., (1994) Proc. Natl. Acad. Sci. USA 91:9564-9568, Emi et al. (1991) Journal of Virology 65:1202-1207). VSV-G protein can be used to pseudotype certain retroviruses because its cytoplasmic tail is capable of interacting with the retroviral cores.

The provision of a non-retroviral pseudotyping envelope such as VSV-G protein gives the advantage that vector particles can be concentrated to a high titre without loss of infectivity (Akkina et al. (1996) J. Virol. 70:2581-5). Retrovirus envelope proteins are apparently unable to withstand the shearing forces during ultracentrifugation, probably because they consist of two non-covalently linked subunits. The interaction between the subunits may be disrupted by the centrifugation. In comparison the VSV glycoprotein is composed of a single unit. VSV-G protein pseudotyping can therefore offer potential advantages for both efficient target cell infection/transduction and during manufacturing processes.

WO 2000/52188 describes the generation of pseudotyped retroviral vectors, from stable producer cell lines, having vesicular stomatitis virus-G protein (VSV-G) as the membrane-associated viral envelope protein, and provides a gene sequence for the VSV-G protein.

### Ross River Virus

The Ross River viral envelope has been used to pseudotype a non-primate lentiviral vector (FIV) and following systemic administration predominantly transduced the liver (Kang et al., 2002, J. Virol., 76:9378-9388). Efficiency was reported to be 20-fold greater than obtained with VSV-G pseudotyped vector, and caused less cytotoxicity as measured by serum levels of liver enzymes suggestive of hepatotoxicity.

### Baculovirus GP64

The baculovirus GP64 protein has been shown to be an alternative to VSV-G for viral vectors used in the large-scale production of high-titre virus required for clinical and commercial applications (Kumar M, Bradow B P, Zimmerberg J (2003) Hum Gene Ther. 14(1):67-77). Compared with VSV-G-pseudotyped vectors, GP64-pseudotyped vectors have a similar broad tropism and similar native titres. Because, GP64 expression does not kill cells, HEK293T-based cell lines constitutively expressing GP64 can be generated.

### Alternative Envelopes

Other envelopes which give reasonable titre when used to pseudotype EIAV include Mokola, Rabies, Ebola and LCMV (lymphocytic choriomeningitis virus). Intravenous infusion into mice of lentivirus pseudotyped with 4070A led to maximal gene expression in the liver.

### Packaging Sequence

As utilized within the context of the present invention the term “packaging signal”, which is referred to interchangeably as “packaging sequence” or “psi”, is used in reference to the non-coding, cis-acting sequence required for encapsidation of retroviral RNA strands during viral particle formation. In HIV-1, this sequence has been mapped to loci extending from upstream of the major splice donor site (SD) to at least the gag start codon (some or all of the 5′ sequence of gag to nucleotide 688 may be included). In EIAV the packaging signal comprises the R region into the 5′ coding region of Gag.

As used herein, the term “extended packaging signal” or “extended packaging sequence” refers to the use of sequences around the psi sequence with further extension into the gag gene. The inclusion of these additional packaging sequences may increase the efficiency of insertion of vector RNA into viral particles.

Feline immunodeficiency virus (FIV) RNA encapsidation determinants have been shown to be discrete and non-continuous, comprising one region at the 5′ end of the genomic mRNA (R-U5) and another region that mapped within the proximal 311 nt of gag (Kaye et al., J Virol. October;69(10):6588-92 (1995).

### Internal Ribosome Entry Site (IRES)

Insertion of IRES elements allows expression of multiple coding regions from a single promoter (Adam et al (as above); Koo et al (1992) Virology 186:669-675; Chen et al 1993 J. Virol 67:2142-2148). IRES elements were first found in the non-translated 5′ ends of picornaviruses where they promote cap-independent translation of viral proteins (Jang et al (1990) Enzyme 44: 292-309). When located between open reading frames in an RNA, IRES elements allow efficient translation of the downstream open reading frame by promoting entry of the ribosome at the IRES element followed by downstream initiation of translation.

A review on IRES is presented by Mountford and Smith (TIG May 1995 vol 11, No 5:179-184). A number of different IRES sequences are known including those from encephalomyocarditis virus (EMCV) (Ghattas, I. R., et al., Mol. Cell. Biol., 11:5848-5859 (1991); BiP protein [Macejak and Sarnow, Nature 353:91 (1991)]; the Antennapedia gene of Drosophila (exons d and e) [Oh, et al., Genes & Development, 6:1643-1653 (1992)] as well as those in polio virus (PV) [Pelletier and Sonenberg, Nature 334: 320-325 (1988); see also Mountford and Smith, TIG 11, 179-184 (1985)].

IRES elements from PV, EMCV and swine vesicular disease virus have previously been used in retroviral vectors (Coffin et al, as above).

The term “IRES” includes any sequence or combination of sequences which work as or improve the function of an IRES. The IRES(s) may be of viral origin (such as EMCV IRES, PV IRES, or FMDV 2A-like sequences) or cellular origin (such as FGF2 IRES, NRF IRES, Notch 2 IRES or EIF4 IRES).

In order for the IRES to be capable of initiating translation of each polynucleotide it should be located between or prior to the polynucleotides in the modular construct.

The nucleotide sequences utilised for development of stable cell lines require the addition of selectable markers for selection of cells where stable integration has occurred. These selectable markers can be expressed as a single transcription unit within the nucleotide sequence or it may be preferable to use IRES elements to initiate translation of the selectable marker in a polycistronic message (Adam et al 1991 J.Virol. 65, 4985).

### Genetic Orientation and Insulators

It is well known that nucleic acids are directional and this ultimately affects mechanisms such as transcription and replication in the cell. Thus genes can have relative orientations with respect to one another when part of the same nucleic acid construct.

In certain aspects of the present invention, at least two nucleic acid sequences present at the same locus in the cell or construct can be in a reverse and/or alternating orientations. In other words, in certain aspects of the invention at this particular locus, the pair of sequential genes will not have the same orientation. This can help prevent both transcriptional and translational read-through when the region is expressed within the same physical location of the host cell.

Having the alternating orientations benefits retroviral vector production when the nucleic acids required for vector production are based at the same genetic locus within the cell. This in turn can also improve the safety of the resulting constructs in preventing the generation of replication-competent retroviral vectors.

When nucleic acid sequences are in reverse and/or alternating orientations the use of insulators can prevent inappropriate expression or silencing of a NOI from its genetic surroundings.

The term “insulator” refers to a class of nucleotide, e.g.DNA, sequence elements that when bound to insulator-binding proteins possess an ability to protect genes from surrounding regulator signals. There are two types of insulators: an enhancer blocking function and a chromatin barrier function. When an insulator is situated between a promoter and an enhancer, the enhancer-blocking function of the insulator shields the promoter from the transcription-enhancing influence of the enhancer (Geyer and Corces 1992; Kellum and Schedl 1992). The chromatin barrier insulators function by preventing the advance of nearby condensed chromatin which would lead to a transcriptionally active chromatin region turning into a transcriptionally inactive chromatin region and resulting in silencing of gene expression. Insulators which inhibit the spread of heterochromatin, and thus gene silencing, recruit enzymes involved in histone modifications to prevent this process (Yang J, Corces VG. 2011; 110:43-76; Huang, Li et al. 2007; Dhillon, Raab et al. 2009). An insulator can have one or both of these functions and the chicken β-globin insulator (cHS4) is one such example. This insulator is the most extensively studied vertebrate insulator, is highly rich in G+C and has both enhancer-blocking and heterochromatic barrier functions (Chung J H, Whitely M, Felsenfeld G. Cell. 1993:74:505-514). Other such insulators with enhancer blocking functions are not limited to but include the following: human β-globin insulator 5 (HS5), human β-globin insulator 1 (HS1), and chicken β-globin insulator (cHS3) (Farrell CM1, West AG, Felsenfeld G., Mol Cell Biol. 2002 June;22(11):3820-31; J Ellis et al. EMBO J. 1996-2-1; 15(3): 562-568). In addition to reducing unwanted distal interactions the insulators also help to prevent promoter interference (i.e. where the promoter from one transcription unit impairs expression of an adjacent transcription unit) between adjacent retroviral nucleic acid sequences. If the insulators are used between each of the retroviral vector nucleic acid sequences, then the reduction of direct read-through will help prevent the formation of replication-competent retroviral vector particles.

The insulator may be present between each of the retroviral nucleic acid sequences. In one aspect, the use of insulators prevents promoter-enhancer interactions from one NOI expression cassette interacting with another NOI expression cassette in a nucleotide sequence encoding vector components.

An insulator may be present between the vector genome and gag-pol sequences. This therefore limits the likelihood of the production of a replication-competent retroviral vector and ‘wild-type’ like RNA transcripts, improving the safety profile of the construct. The use of insulator elements to improve the expression of stably integrated multigene vectors is cited in Moriarity et al, Nucleic Acids Res. 2013 April;41(8):e92.

### Vector Titre

The skilled person will understand that there are a number of different methods of determining the titre of lentiviral vectors. Titre is often described as transducing units/mL (TU/mL). Titre may be increased by increasing the number of vector particles and by increasing the specific activity of a vector preparation.

### Clarification

Once the viral preparation is harvested from a cell culture as a supernatant, the supernatant may be clarified. The clarified supernatant may be used as the viral vector preparation that is passed through the cation exchange column.

Any suitable method of clarification may be employed.

For example, clarification may be performed by using a filter to remove cell debris and other impurities. Suitable filters may utilize cellulose filters, regenerated cellulose fibers, cellulose fibers combined with inorganic filter aids (e.g. diatomaceous earth, perlite, fumed silica), cellulose filters combined with inorganic filter aids and organic resins, or any combination thereof, and polymeric filters (examples include but are not limited to nylon, polypropylene, polyethersulfone) to achieve effective removal and acceptable recoveries. In general, a multiple stage process is preferable but not required. An exemplary two or three-stage process would consist of a coarse filter(s) to remove large precipitate and cell debris followed by polishing second stage filter(s) with nominal pore sizes greater than 0.2 micron but less than 1 micron. The optimal combination may be a function of the precipitate size distribution as well as other variables. In addition, single stage operations employing a relatively small pore size filter or centrifugation may also be used for clarification. More generally, any clarification approach including but not limited to dead-end filtration, microfiltration, centrifugation, or body feed of filter aids (e.g. diatomaceous earth) in combination with dead-end or depth filtration, which provides a filtrate of suitable clarity to not foul the membrane and/or resins in the subsequent steps, will be acceptable to use in the clarification step of the present invention.

In one embodiment, depth filtration and membrane filtration is used. Commercially available products useful in this regard are for instance mentioned in WO 03/097797, p. 20-21. Membranes that can be used may be composed of different materials, may differ in pore size, and may be used in combinations. They can be commercially obtained from several vendors.

Preferably the filter used for clarification is in the range of 1.2 to 0.22 μm.

More preferably the filter used for clarification is either a 1.2/0.45 μm filter or an asymmetric filter with a minimum nominal pore size of 0.22 μm.

### Nucleotide of Interest

The viral vectors described herein typically comprise a viral genome that encodes a transgene (also referred to herein as a “nucleotide of interest” or a NOI).

The NOI may be introduced into a target cell using a viral vector of the present invention. In this context, a “target cell” is a cell in which it is desired to express the NOI. Delivery to the target cell may be performed in vivo, ex vivo or in vitro.

In a preferred embodiment, the nucleotide of interest gives rise to a therapeutic effect.

The NOI may have a therapeutic or diagnostic application. Suitable NOIs include, but are not limited to sequences encoding enzymes, co-factors, cytokines, chemokines, hormones, antibodies, anti-oxidant molecules, engineered immunoglobulin-like molecules, single chain antibodies, fusion proteins, immune co-stimulatory molecules, immunomodulatory molecules, chimeric antigen receptors a transdomain negative mutant of a target protein, toxins, conditional toxins, antigens, transcription factors, structural proteins, reporter proteins, subcellular localization signals, tumour suppressor proteins, growth factors, membrane proteins, receptors, vasoactive proteins and peptides, anti-viral proteins and ribozymes, and derivatives thereof (such as derivatives with an associated reporter group). The NOIs may also encode micro-RNA.

In one embodiment, the NOI may be useful in the treatment of a neurodegenerative disorder.

In another embodiment, the NOI may be useful in the treatment of Parkinson's disease and multiple system atrophy.

In another embodiment, the NOI may encode an enzyme or enzymes involved in dopamine synthesis. For example, the enzyme may be one or more of the following: tyrosine hydroxylase, GTP-cyclohydrolase I and/or aromatic amino acid dopa decarboxylase. The sequences of all three genes are available (GenBank® Accession Nos. X05290, U19523 and M76180, respectively).

In another embodiment, the NOI may encode the vesicular monoamine transporter 2 (VMAT2). In an alternative embodiment the viral genome may comprise a NOI encoding aromatic amino acid dopa decarboxylase and a NOI encoding VMAT2. Such a genome may be used in the treatment of Parkinson's disease, in particular in conjunction with peripheral administration of L-DOPA.

In another embodiment the NOI may encode a therapeutic protein or combination of therapeutic proteins.

In another embodiment, the NOI may encode a protein or proteins selected from the group consisting of glial cell derived neurotophic factor (GDNF), brain derived neurotrophic factor (BDNF), ciliary neurotrophic factor (CNTF), neurotrophin-3 (NT-3), acidic fibroblast growth factor (aFGF), basic fibroblast growth factor (bFGF), interleukin-1 beta (IL-1β), tumor necrosis factor alpha (TNF-α), insulin growth factor-2, VEGF-A, VEGF-B, VEGF-C/VEGF-2, VEGF-D, VEGF-E, PDGF-A, PDGF-B, hetero- and homo-dimers of PDFG-A and PDFG-B.

In another embodiment, the NOI may encode an anti-angiogenic protein or anti-angiogenic proteins selected from the group consisting of angiostatin, endostatin, platelet factor 4, pigment epithelium derived factor (PEDF), placental growth factor, restin, interferon-α, interferon-inducible protein, gro-beta and tubedown-1, interleukin(IL)-1, IL-12, retinoic acid, anti-VEGF antibodies or fragments/variants thereof such as aflibercept, thrombospondin, VEGF receptor proteins such as those described in U.S. Pat. Nos. 5,952,199 and 6,100,071, and anti-VEGF receptor antibodies.

In another embodiment, the NOI may encode anti-inflammatory proteins, antibodies or fragment/variants of proteins or antibodies selected from the group consisting of NF-kB inhibitors, IL1beta inhibitors, TGFbeta inhibitors, IL-6 inhibitors, IL-23 inhibitors, IL-18 inhibitors, Tumour necrosis factor alpha and Tumour necrosis factor beta, Lymphotoxin alpha and Lymphotoxin beta, LIGHT inhibitors, alpha synuclein inhibitors, Tau inhibitors, beta amyloid inhibitors, IL-17 inhibitors, IL-33 inhibitors, IL-33 receptor inhibitors, and TSLP inhibitors.

In another embodiment the NOI may encode cystic fibrosis transmembrane conductance regulator (CFTR).

In another embodiment the NOI may encode a protein normally expressed in an ocular cell.

In another embodiment, the NOI may encode a protein normally expressed in a photoreceptor cell and/or retinal pigment epithelium cell.

In another embodiment, the NOI may encode a protein selected from the group comprising RPE65, arylhydrocarbon-interacting receptor protein like 1 (AIPL1), CRB1, lecithin retinal acetyltransferace (LRAT), photoreceptor-specific homeo box (CRX), retinal guanylate cyclise (GUCY2D), RPGR interacting protein 1 (RPGRIP1), LCA2, LCA3, LCA5, dystrophin, PRPH2, CNTF, ABCR/ABCA4, EMP1, TIMP3, MERTK, ELOVL4, MYO7A, USH2A, VMD2, RLBP1, COX-2, FPR, harmonin, Rab escort protein 1, CNGB2, CNGA3, CEP 290, RPGR, RS1, RP1, PRELP, glutathione pathway enzymes and opticin.

In other embodiments, the NOI may encode the human clotting Factor VIII or Factor IX.

In other embodiments, the NOI may encode protein or proteins involved in metabolism selected from the group comprising phenylalanine hydroxylase (PAH), Methylmalonyl CoA mutase, Propionyl CoA carboxylase, Isovaleryl CoA dehydrogenase, Branched chain ketoacid dehydrogenase complex, Glutaryl CoA dehydrogenase, Acetyl CoA carboxylase, propionyl CoA carboxylase, 3 methyl crotonyl CoA carboxylase, pyruvate carboxylase, carbamoyl-phophate synthase ammonia, ornithine transcarbamylase, alpha galactosidase A, glucosylceramidase beta, cystinosin, glucosamine(N-acetyl)-6-sulfatase, N-acetyl-alpha-glucosaminidase, glucose-6-phosphatase, ATP7B, ATP8B1, ABCB11, ABCB4, TJP2, N-sulfoglucosamine sulfohydrolase, Galactosamine-6 sulfatase, arylsulfatase A, cytochrome B-245 beta, ABCD1, ornithine carbamoyltransferase, argininosuccinate synthase, argininosuccinate lysase, arginase 1, alanine glycoxhylate amino transferase, ATP-binding cassette, sub-family B members.

In other embodiments, the NOI may encode a chimeric antigen receptor (CAR) or a T cell receptor (TCR). In one embodiment, the CAR is an anti-5T4 CAR. In other embodiments, the NOI may encode B-cell maturation antigen (BCMA), CD19, CD22, CD20, CD138, CD30, CD33, CD123, CD70, prostate specific membrane antigen (PSMA), Lewis Y antigen (LeY), Tyrosine-protein kinase transmembrane receptor (ROR1), Mucin 1, cell surface associated (Muc1), Epithelial cell adhesion molecule (EpCAM), endothelial growth factor receptor (EGFR), insulin, protein tyrosine phosphatase, non-receptor type 22, interleukin 2 receptor, alpha, interferon induced with helicase C domain 1, human epidermal growth factor receptor (HER2), glypican 3 (GPC3), disialoganglioside (GD2), mesiothelin, vesicular endothelial growth factor receptor 2 (VEGFR2). Smith antigen, Ro60, double stranded DNA, phospholipids, proinsulin, insulinoma antigen 2 (IA-2), 65 kDa isoform of glutamic acid decarboxylase (GAD65), chromogranin A (CHGA), islet amyloid polypeptide (IAPP), islet-specific glucose-6-phosphatase catalytic subunit-related protein (IGRP), zinc transporter 8 (ZnT8).

In other embodiments, the NOI may encode a chimeric antigen receptor (CAR) against NKG2D ligands selected from the group comprising ULBP1, 2 and 3, H60, Rae-1a, b, g, d, MICA, MICB.

In further embodiments the NOI may encode SGSH, SUMF1, GAA, the common gamma chain (CD132), adenosine deaminase, WAS protein, globins, alpha galactosidase A, 5-aminolevulinate (ALA) synthase, 5-aminolevulinate dehydratase (ALAD), Hydroxymethylbilane (HMB) synthase, Uroporphyrinogen (URO) synthase, Uroporphyrinogen (URO) decarboxylase, Coproporphyrinogen (COPRO) oxidase, Protoporphyrinogen (PROTO) oxidase, Ferrochelatase, α-L-iduronidase, Iduronate sulfatase, Heparan sulfamidase, N-acetylglucosaminidase, Heparan-α-glucosaminide N-acetyltransferase, 3 N-acetylglucosamine 6-sulfatase, Galactose-6-sulfate sulfatase, β-galactosidase, N-acetylgalactosamine-4-sulfatase, β-glucuronidase and Hyaluronidase.

In addition to the NOI the vector may also comprise or encode a siRNA, shRNA, or regulated shRNA. (Dickins et al. (2005) Nature Genetics 37: 1289-1295, Silva et al. (2005) Nature Genetics 37:1281-1288).

### Indications

The vectors, including retroviral and AAV vectors, purified according to the method of the method disclosure may be used to deliver one or more NOI(s) useful in the treatment of the disorders listed in WO 1998/05635, WO 1998/07859, WO 1998/09985. The nucleotide of interest may be DNA or RNA. Examples of such diseases are given below:

A disorder which responds to cytokine and cell proliferation/differentiation activity; immunosuppressant or immunostimulant activity (e.g. for treating immune deficiency, including infection with human immunodeficiency virus, regulation of lymphocyte growth; treating cancer and many autoimmune diseases, and to prevent transplant rejection or induce tumour immunity); regulation of haematopoiesis (e.g. treatment of myeloid or lymphoid diseases); promoting growth of bone, cartilage, tendon, ligament and nerve tissue (e.g. for healing wounds, treatment of burns, ulcers and periodontal disease and neurodegeneration); inhibition or activation of follicle-stimulating hormone (modulation of fertility); chemotactic/chemokinetic activity (e.g. for mobilising specific cell types to sites of injury or infection); haemostatic and thrombolytic activity (e.g. for treating haemophilia and stroke); anti-inflammatory activity (for treating, for example, septic shock or Crohn's disease); macrophage inhibitory and/or T cell inhibitory activity and thus, anti-inflammatory activity; anti-immune activity (i.e. inhibitory effects against a cellular and/or humoral immune response, including a response not associated with inflammation); inhibition of the ability of macrophages and T cells to adhere to extracellular matrix components and fibronectin, as well as up-regulated fas receptor expression in T cells.

Malignancy disorders, including cancer, leukaemia, benign and malignant tumour growth, invasion and spread, angiogenesis, metastases, ascites and malignant pleural effusion.

Autoimmune diseases including arthritis, including rheumatoid arthritis, hypersensitivity, psoriasis, Sjogren's syndrome, allergic reactions, asthma, chronic obstructive pulmonary disease, systemic lupus erythematosus, Type 1 diabetes mellitus, Crohn's disease, ulcerative colitis, collagen diseases and other diseases.

Vascular diseases including arteriosclerosis, atherosclerotic heart disease, reperfusion injury, cardiac arrest, myocardial infarction, vascular inflammatory disorders, respiratory distress syndrome, cardiovascular effects, peripheral vascular disease, migraine and aspirin-dependent anti-thrombosis, stroke, cerebral ischaemia, ischaemic heart disease or other diseases.

Diseases of the gastrointestinal tract including peptic ulcer, ulcerative colitis, Crohn's disease and other diseases.

Hepatic diseases including hepatic fibrosis, liver cirrhosis, amyloidosis.

Inherited metabolic disorders including phenylketonuria PKU, Wilson disease, organic acidemias, glycogen storage diseases, urea cycle disorders, cholestasis, and other diseases, or other diseases.

Renal and urologic diseases including thyroiditis or other glandular diseases, glomerulonephritis, lupus nephritis or other diseases.

Ear, nose and throat disorders including otitis or other oto-rhino-laryngological diseases, dermatitis or other dermal diseases.

Dental and oral disorders including periodontal diseases, periodontitis, gingivitis or other dental/oral diseases.

Testicular diseases including orchitis or epididimo-orchitis, infertility, orchidal trauma or other testicular diseases.

Gynaecological diseases including placental dysfunction, placental insufficiency, habitual abortion, eclampsia, pre-eclampsia, endometriosis and other gynaecological diseases.

Ophthalmologic disorders such as Leber Congenital Amaurosis (LCA) including LCA10, posterior uveitis, intermediate uveitis, anterior uveitis, conjunctivitis, chorioretinitis, uveoretinitis, optic neuritis, glaucoma, including open angle glaucoma and juvenile congenital glaucoma, intraocular inflammation, e.g. retinitis or cystoid macular oedema, sympathetic ophthalmia, scleritis, retinitis pigmentosa, macular degeneration including age related macular degeneration (AMD) and juvenile macular degeneration including Best Disease, Best vitelliform macular degeneration, Stargardt's Disease, Usher's syndrome, Doyne's honeycomb retinal dystrophy, Sorby's Macular Dystrophy, Juvenile retinoschisis, Cone-Rod Dystrophy, Corneal Dystrophy, Fuch's Dystrophy, Leber's congenital amaurosis, Leber's hereditary optic neuropathy (LHON), Adie syndrome, Oguchi disease, degenerative fondus disease, ocular trauma, ocular inflammation caused by infection, proliferative vitreo-retinopathies, acute ischaemic optic neuropathy, excessive scarring, e.g. following glaucoma filtration operation, reaction against ocular implants, corneal transplant graft rejection, and other ophthalmic diseases, such as diabetic macular oedema, retinal vein occlusion, RLBP1-associated retinal dystrophy, choroideremia and achromatopsia.

Neurological and neurodegenerative disorders including Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, strokes, post-polio syndrome, psychiatric disorders, myelitis, encephalitis, subacute sclerosing pan-encephalitis, encephalomyelitis, acute neuropathy, subacute neuropathy, chronic neuropathy, Fabry disease, Gaucher disease, Cystinosis, Pompe disease, metachromatic leukodystrophy, Wiscott Aldrich Syndrome, adrenoleukodystrophy, beta-thalassemia, sickle cell disease, Guillaim-Barre syndrome, Sydenham chorea, myasthenia gravis, pseudo-tumour cerebri, Down's Syndrome, Huntington's disease, Frontotemporal dementia, CNS compression or CNS trauma or infections of the CNS, muscular atrophies and dystrophies, diseases, conditions or disorders of the central and peripheral nervous systems, motor neuron disease including amyotropic lateral sclerosis, spinal muscular atropy, spinal cord and avulsion injury.

Other diseases and conditions such as cystic fibrosis, mucopolysaccharidosis including Sanfilipo syndrome A, Sanfilipo syndrome B, Sanfilipo syndrome C, Sanfilipo syndrome D, Hunter syndrome, Hurler-Scheie syndrome, Morquio syndrome, ADA-SCID, X-linked SCID, X-linked chronic granulomatous disease, porphyria, haemophilia A, haemophilia B, post-traumatic inflammation, haemorrhage, coagulation and acute phase response, cachexia, anorexia, acute infection, septic shock, infectious diseases, diabetes mellitus, complications or side effects of surgery, bone marrow transplantation or other transplantation complications and/or side effects, complications and side effects of gene therapy, e.g. due to infection with a viral carrier, or AIDS, to suppress or inhibit a humoral and/or cellular immune response, for the prevention and/or treatment of graft rejection in cases of transplantation of natural or artificial cells, tissue and organs such as cornea, bone marrow, organs, lenses, pacemakers, natural or artificial skin tissue.

siRNA, Micro-RNA and shRNA

In certain other embodiments, the NOI comprises a micro-RNA. Micro-RNAs are a very large group of small RNAs produced naturally in organisms, at least some of which regulate the expression of target genes. Founding members of the micro-RNA family are let-7 and lin-4. The let-7 gene encodes a small, highly conserved RNA species that regulates the expression of endogenous protein-coding genes during worm development. The active RNA species is transcribed initially as an ˜70 nt precursor, which is post-transcriptionally processed into a mature ˜21 nt form. Both let-7 and lin-4 are transcribed as hairpin RNA precursors which are processed to their mature forms by Dicer enzyme.

In addition to the NOI the vector may also comprise or encode a siRNA, shRNA, or regulated shRNA (Dickins et al. (2005) Nature Genetics 37: 1289-1295, Silva et al. (2005) Nature Genetics 37:1281-1288).

Post-transcriptional gene silencing (PTGS) mediated by double-stranded RNA (dsRNA) is a conserved cellular defence mechanism for controlling the expression of foreign genes. It is thought that the random integration of elements such as transposons or viruses causes the expression of dsRNA which activates sequence-specific degradation of homologous single-stranded mRNA or viral genomic RNA. The silencing effect is known as RNA interference (RNAi) (Ralph et al. (2005) Nature Medicine 11:429-433). The mechanism of RNAi involves the processing of long dsRNAs into duplexes of about 21-25 nucleotide (nt) RNAs. These products are called small interfering or silencing RNAs (siRNAs) which are the sequence-specific mediators of mRNA degradation. In differentiated mammalian cells, dsRNA >30 bp has been found to activate the interferon response leading to shut-down of protein synthesis and non-specific mRNA degradation (Stark et al., Annu Rev Biochem 67:227-64 (1998)). However this response can be bypassed by using 21 nt siRNA duplexes (Elbashir et al., EMBO J. December 3;20(23):6877-88 (2001), Hutvagner et al., Science.August 3, 293(5531):834-8. Eupub July 12 (2001)) allowing gene function to be analysed in cultured mammalian cells.

### FIGURES AND EXAMPLES

These and other aspects of the present invention will now be described, by way of example, with reference to the accompanying Figures and Examples.

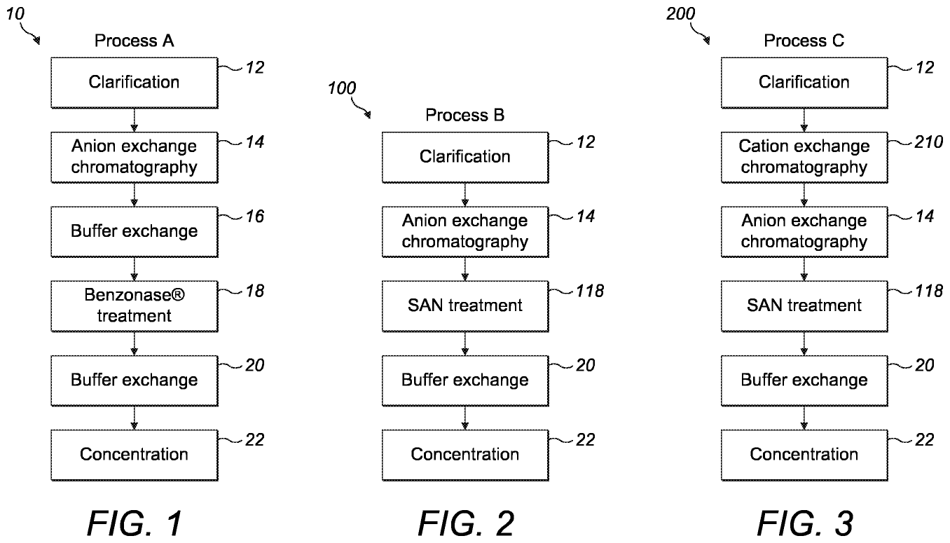

FIG. 1 is a flowchart 10 of an example of a prior art process for purifying a viral vector preparation (Process A).

Viral vector may be produced by culturing cells in a bioreactor (not shown). Once the viral vector is ready for harvest, the cell medium may be subjected to nucleic acid cleavage in a bioreactor.

In some examples, the cell culture may be treated with an endonuclease, for example, Benzonase @Nuclease in a bioreactor in the presence of a co-factor. A suitable co-factor may be magnesium chloride. The endonuclease may be incubated in the bioreactor for, for example, 60 to 120 minutes at 36 to 38 degrees C.

The endonuclease may cleave some of the DNA in the bioreactor. The cell culture may then be harvested and the harvested cells clarified 12. Clarification may be performed by passing the sample through a 10 μm filter, for example, to remove cells. The filtrate may then be passed through a 0.2 μm filter to clarify the sample.

The sample may then be treated by anion exchange chromatography 14. Anion exchange chromatography is a form of ion exchange chromatography, which is used to separate molecules based on their net surface charge. Anion exchange chromatography uses an anion exchanger (i.e. a positively charged ion exchanger) with an affinity for molecules having net negative surface charges. In some examples, the anion exchanger used in the anion exchange chromatography step 14 may be an anion exchanger comprising quaternary ammonium groups. An example of such a resin is Sartobind Q@.

As the viral vector in the sample is negatively charged, viral vector particles can bind to the anion exchanger and more positively charged molecules flow through. The bound viral vector particles can then be eluted by flushing the anion exchanger with a buffer, for example, a high salt buffer. Although the viral vector may selectively bind to the positively charged sites on the anion exchanger, other negatively charged species including, for example, DNA from the host cell may also bind to the anion exchange resin. It has also been found that, despite their positive charge, aggregating species, such as histones, may also bind to the anion exchange resin. The reasons for this are not well understood. However, it is possible that aggregates of e.g. histones and negatively charged species, such as DNA, may form. The resulting aggregates may have sufficient negative charge to bind to the anion exchanger.

When the anion exchange resin is eluted, the bound species are eluted as eluent. This eluent may be subjected to a nucleic acid cleavage 18 using Benzonase® nuclease and magnesium chloride as co-factor. As discussed above, the activity of Benzonase® nuclease may be inhibited by the high salt concentrations of the buffer present in the eluent from the anion exchanger. Accordingly, the eluent may be subjected to a buffer exchange 16 prior to nucleic acid cleavage 18 by Benzonase® nuclease treatment. A further buffer exchange step 20 may be performed, prior to concentration 22 of the viral vector.

FIG. 2 is a flowchart of an example process for purifying a viral vector preparation (Process B). The process is similar to the process shown in FIG. 1 and like steps have been denoted with like numerals. However, in the flowchart 100 of FIG. 2, the buffer exchange step 16 prior to nucleic acid cleavage has been omitted and the eluent from anion exchanger 14 subjected to nucleic acid cleavage using a halotolerant and/or halophilic nuclease (e.g. Salt Active Nuclease) 118 instead of Benzonase® Nuclease 18.

As explained above, halotolerant and/or halophilic nucleases such as Salt Active Nucleases are active under high salt concentrations. Accordingly, it may be possible to treat the eluate from the anion exchanger using such nucleases without pre-treating the eluate by a salt-removal step (e.g. buffer exchange). This can reduce the complexity of the process, improving process efficiency. Furthermore, high salt conditions can, at least in certain cases, promote dissociation of histone-DNA complexes, for example, to unbound DNA and hydrophilic agglutinates. This can result in reduced agglomeration, which may be beneficial for the efficacy of downstream filtration steps. Furthermore, the unbound DNA may be more accessible to cleavage by the nuclease (e.g. Salt Active Nuclease). This can improve the efficacy of DNA removal.

Following halotolerant/halophilic nuclease treatment (e.g. Salt Active Nuclease treatment) 116, the vector may be further treated by buffer exchange 20 and concentrated 22.

FIG. 3 is a flowchart 200 of an example process for purifying a viral vector preparation according to an example of the present invention (Process C). The process is similar to the process shown in FIG. 2 and like steps are denoted using like numerals. However, in the flowchart of FIG. 3, a cation exchange step 210 is performed on the clarified viral vector preparation treated in step 12 and prior to anion exchange 14.

For example, the cation exchanger may be a cation exchange resin comprising sulfonic acid groups. An example of such a resin is Sartobind S®.

Where a cation exchange membrane is used, the cation exchange membrane may be treated with a suitable buffer to, for example, hydrate the membrane and/or flush out any leachable components that may be bound to the membrane. Any suitable chromatography buffer may be used in this step.

The membrane may then be sanitized, for example, with a suitable alkali solution. The membrane may then be flushed again with a suitable buffer. Any suitable chromatography buffer may be used in this step.

A high salt buffer may then be passed across the membrane to “charge” the functional groups on the cation exchange membrane. The membrane may then be equilibrated with a suitable chromatography buffer.

Following equilibration, the viral vector preparation may be contacted with the membrane. The viral preparation may be clarified supernatant from the clarification step 12.

As explained above, DNA from e.g. host cells may interact or form complexes with e.g. positively charged species in the viral vector preparation, including, for example, histones. This interaction and/or complex-formation may allow DNA or DNA-containing complexes to bind to the cation exchanger, facilitating the separation of at least some DNA from the viral vector preparation by cation exchange. The flow-through treated by anion exchange, therefore, may contain reduced levels of DNA. Accordingly, by passing a viral vector preparation through a cation exchanger, improved separation of e.g. DNA impurities from the host cell can be achieved.

Furthermore, by passing a viral vector preparation through a cation exchanger, the risk of particle aggregation may also be reduced. Excessive particle aggregation can be detrimental to the throughput of any downstream filtration steps, for example, by prematurely restricting or blocking the pores of filtration membranes. It has been found that cation exchange can be used to remove aggregating species, including, for example, histones from the viral vector preparation. As histones can induce particle aggregation, it may be possible to improve the efficacy of downstream filtration by passing a viral vector preparation through a cation exchanger and contacting flow-through from the cation exchanger with an anion exchanger.

Examples of DNA-containing complexes or aggregates that may be present or form in the viral vector preparation are chromatin hetero aggregates (approximately 20-400 nm). Chromatin is a complex of DNA and host cell protein, with the basic structural unit of chromatin being a nucleosome (DNA wrapped around host cell histone proteins). Such aggregates can exacerbate aggregation. For example, such aggregates can associate with larger bodies such as nucleosome arrays, and serve as nucleation sites for secondary agglomerations. They may also bind to virus species. Co-concentration of these species may lead to enhanced aggregation and reduced product quality with a potential loss of infective vector titre from aggregate to virus binding. However, because of the positive nature of the histone portion of the aggregate, such aggregates may bind to the cation exchanger in step 210.

### EXAMPLES

### Example 1—Particle Size Measurements Using Dynamic Light Scattering Quantification (DLS)

### Bioreactor Experimental Conditions

Two 5 L bioreactors were employed in this study using a cationic lipid-based transfection method. Both bioreactors were transiently transfected with an HIV-1-vector genome encoding 5T4-CAR. This therapeutic vector is for the genetic modification of T cells enabling them to target and kill tumour cells expressing the antigen 5T4 (Owens, LG, Sheard, V E et al J Immunother. 2018-4-41(3): 130-140).

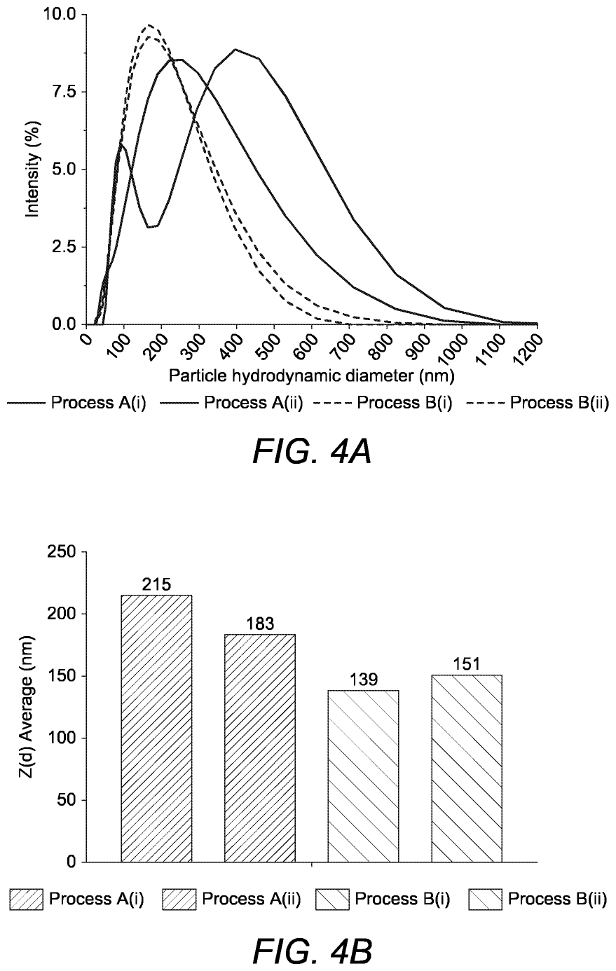

Clarified harvest material was either processed using the down-stream procedure described in relation to FIG. 1 (Process A), using Benzonase® Nuclease, or using SAN in accordance with the down-stream procedure described in relation to FIG. 2 (Process B). FIG. 4A show the difference in particle size distribution between purified vector processed using Benzonase® (BEN) or SAN. The SAN processed vector preparations showed a much tighter particle size distribution indicating a decrease in aggregation.

FIG. 4B displays the Z(d) average particle size for each of the vector preparations with SAN processed vector (Process B) giving a significantly lower particle size compared to Benzonase® treated vector (Process A).

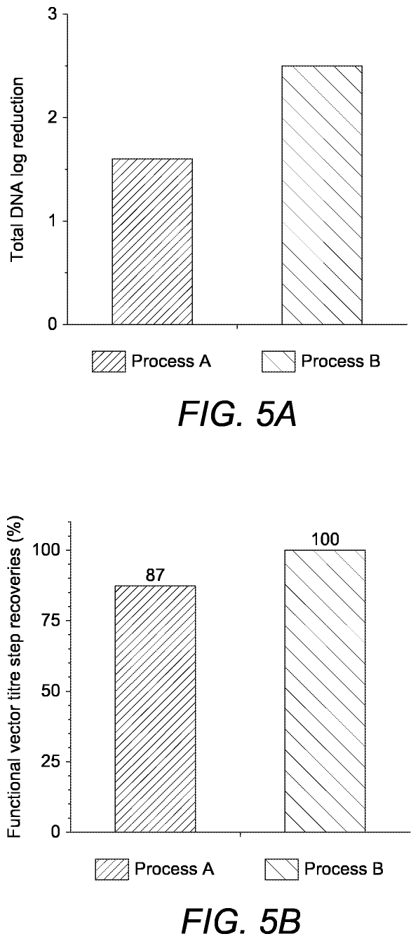

The final preparation of the vector involved pumping the vector through a 0.2 μm sterilizing filter before aliquoting. Samples from the vector were taken and residual DNA quantification was measured. Data shown in FIG. 5A demonstrates the impact of Process B using SAN treatment in reducing residual DNA levels (a log reduction of 2.5 was achieved for Process B v compared to 1.6 for Process A). In addition, due to the reduction in particle size, the percentage recovery (functional vector titre step recovery) is also increased (FIG. 5B) and no vector is being lost during filtration.

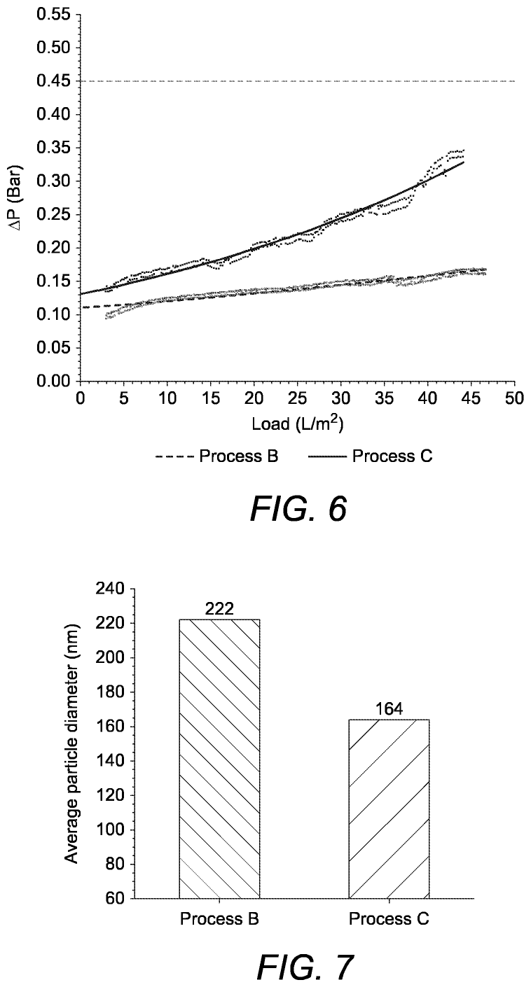

In addition, pressure profiles taken during downstream sterile filtration show that the pressure is reduced when SAN is employed during the down-stream process compared to when Benzonase® is used (see FIG. 6). FIG. 6 shows the positive impact of SAN (Process B) in reducing the pressure profile across the sterile filtration membrane compared to Benzonase® (Process A). This data indicates that vector purified using SAN in the process (Process B) has lower levels of aggregation and therefore reduced pressure is observed.

### Example 2: Impact of Introducing a Cationic Exchange Chromatography Step, Flow Through Mode, Prior to Anion Exchange Chromatography

### Bioreactor Experimental Conditions

Two 5 litre bioreactors were employed in this study using a cationic lipid-based transfection method. Both bioreactors were transiently transfected with an HIV-1-vector genome encoding 5T4-CAR. This therapeutic vector is for the genetic modification of T cells enabling them to target and kill tumour cells expressing the antigen 5T4 (Owens, LG, Sheard, V E et al J Immunother. 2018-4-41(3): 130-140).

Clarified harvest material was either processed using the procedure described with reference to FIG. 2 (Process B), or processed using the procedure described with reference to FIG. 3 (Process C). FIG. 7 shows the impact of purifying vector using the procedure described with reference to FIG. 3 (Process C) compared to Process B. Process C leads to a significant reduction in particle size (measured by dynamic light scattering (DLS)) compared to Process B. This indicates that effect of the cationic exchange chromatography step is a reduction in aggregation.

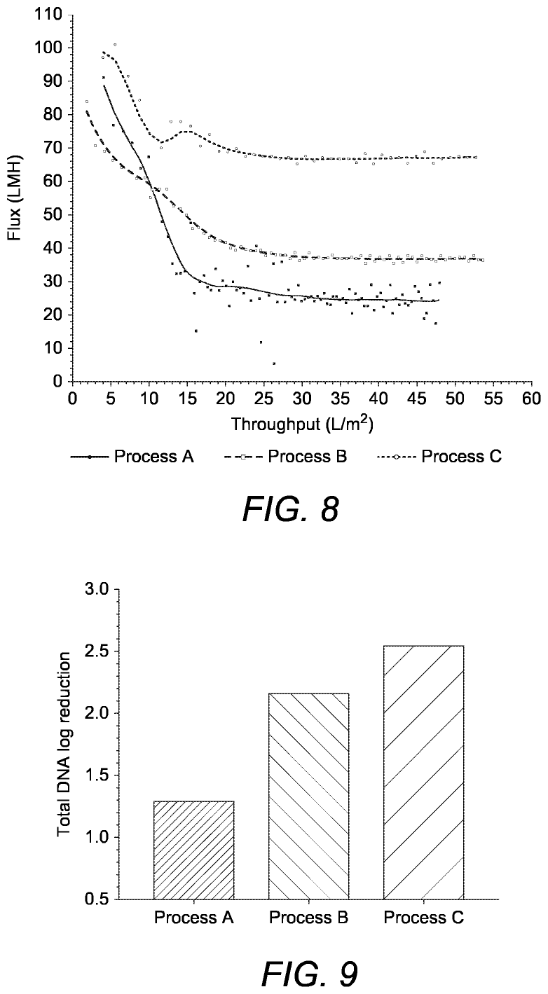

FIG. 8 compares the pressure profiles across the membranes used in the buffer exchange step 20 in Processes A, B and C. This figure further demonstrates the positive impact of Process C down-stream operations in reducing aggregation. When vector is purified using Process A, the pressure profile is higher indicating membrane fouling whereas the pressure is reduced when vector is purified using Process B and is further reduced when vector is purified using Process C.

Moreover, overall residual levels are further decreased when vector is purified using Process C (FIG. 9). Purifying vector using the down-stream Process C leads to a significant reduction in total DNA compared to vector purified using Process B and Process A. This data also illustrates the cumulative impact of combining cationic exchange chromatography with the implementation of SAN following anion exchange chromatography compared to SAN only (Process B).

### Example 3—Mass Spectrometry Analysis Comparison of Vector Purified Using Either Process B or Process C.

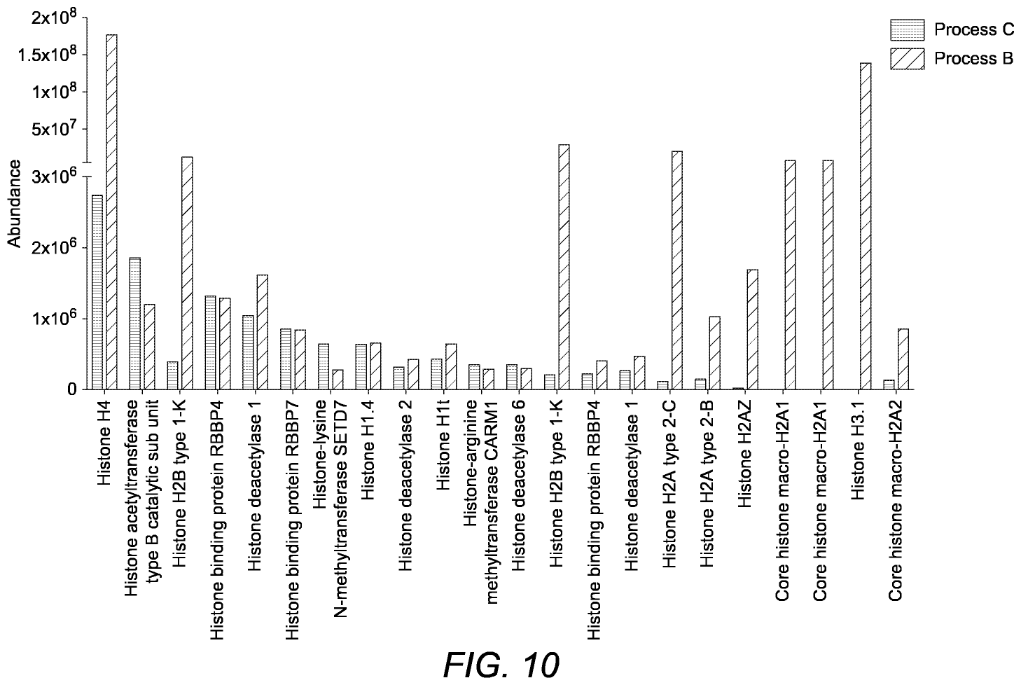

Purified vector obtained either from Process B processed material or Process C was analysed by mass spectrometry and a protein search for all human proteins was performed. Significantly different proteins were selected based on >10-fold change and p value threshold of 0.05. 57 significantly different proteins were identified (FIG. 10). Main change in abundance occurred in histone proteins; ribosomal proteins and DNA/RNA binding proteins. The data suggests that including the cationic exchange chromatography step in the down-stream process is reducing the abundance of histones in the final vector product. The data also supports the hypothesis that this step is reducing DNA levels by capturing histones and residual DNA bound to the histone.

1. A method of purifying a viral vector preparation, said method comprising
passing a viral vector preparation through a cation exchange column,
contacting flow-through from the cation exchange column with an anion exchanger that binds the viral vector, and
eluting the bound viral vector from the anion exchanger as a viral vector eluate.

2. A method as claimed in claim 1, wherein the viral vector is selected from the group consisting of: a retroviral vector, an adenoviral vector, an adeno-associated viral vector, a herpes simplex viral vector, a vaccinia viral vector, a picornaviral vector, and an alphaviral vector.

3. A method as claimed in claim 1 or 2, wherein the viral vector is a retroviral vector.

4. A method as claimed in claim 3, wherein the retroviral vector is a lentiviral vector.

5. A method as claimed in claim 4, wherein the lentiviral vector is derived from a HIV-1, HIV- 2, SIV, FIV, BIV, EIAV, CAEV or visna lentivirus.

6. A method as claimed any one of the preceding claims, wherein the viral vector preparation comprises DNA, and wherein at least some DNA is bound to the cation exchange column when the viral vector preparation is passed through the cation exchange column.

7. A method as claimed in claim 6, wherein the viral vector preparation comprises histone and DNA, and wherein at least some DNA and histone is bound to the cation exchange column when the viral vector preparation is passed through the cation exchange column.

8. A method as claimed in any one of the preceding claim, wherein the flow-through from the cation exchange column is contacted directly with the anion exchanger.

9. A method as claimed in any one of the preceding claims, wherein the viral vector eluate is treated using a halotolerant endonuclease and/or a halophilic endonuclease.

10. A method as claimed in claim 9, wherein the viral vector eluate is treated using salt active nuclease.

11. A method as claimed in claim 8 or 9, wherein, once treated with the endonuclease, the viral vector eluate is filtered.

12. A method as claimed in claim 11, wherein filtration is carried out by tangential flow filtration.

13. A method as claimed in any one of the preceding claims, wherein the cation exchange column comprises a sulfonic acid cation exchanger.

14. A method as claimed in any one of the preceding claims, wherein the anion exchanger comprises a quaternary ammonium anion exchanger.

15. A method as claimed in any one of the preceding claims, wherein the viral vector preparation is produced by culturing cells that produce the viral vector and harvesting a viral vector-containing supernatant from the cell culture.

16. A method as claimed in claim 15, wherein the supernatant harvested from the cell culture is clarified.
